(19) United States
(12) Patent Application Publication
(10) Pub. No.: US 2025/0129068 A1
(43) Pub. Date: Apr. 24, 2025

(54) 4-(2-PYRAZOLO[3,4-B]PYRIDINE-5-YL)ETHYNYL-2-PYRIDINE DERIVATIVES USEFUL AS GCN2 INHIBITORS

(71) Applicant: IP2IPO INNOVATIONS LIMITED, London (GB)

(72) Inventors: Gavin WHITLOCK, Deal Kent (GB); Matthew FUCHTER, London (GB)

(73) Assignee: IP2IPO INNOVATIONS LIMITED, London (GB)

(21) Appl. No.: 18/718,996

(22) PCT Filed: Dec. 13, 2022
(86) PCT No.: PCT/GB2022/053202

§ 371 Date: Nov. 14, 2024

(30) Foreign Application Priority Data
Dec. 14, 2021 (GB) 2118096.3

(51) Int. Cl. C07D 471/04; A61K 31/444; A61K 45/06

(52) U.S. Cl. CPC C07D 471/04; A61K 31/444; A61K 45/06

(57) ABSTRACT

The invention provides compounds of formula:

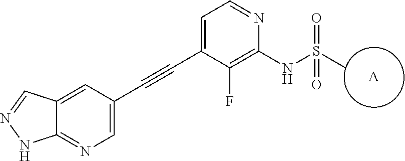

Wherein the substituents are as set out in further detail in the specification. The compounds are potent inhibitors of GCN2 and they have excellent pharmacokinetic properties. The compounds are useful for the treatment or prevention of a variety of conditions, particularly cancer. The invention further provides pharmaceutical compositions comprising the compounds of the invention and uses of the compounds and the compositions.

### FIELD OF THE INVENTION

The present invention relates to compounds of formula (I) and pharmaceutical compositions thereof, and their use as medicaments. The compounds of the invention are inhibitors of general control nonderepressible 2 (GCN2) and as such may be useful for the treatment or prevention of a variety of conditions, and particularly for use in the treatment of diseases, such as cancer.

### BACKGROUND

The kinase general control nonderepressible 2 (GCN2), encoded by EIF2AK4, is a pivotal regulator of cellular adaptations to amino acid shortages (Castilho, B. A., et al (2014) Biochim Biophys Acta 1843, 1948-1968). GCN2 is activated when uncharged tRNAs accumulate as a consequence of low amino acid levels (Romano, P. R., et al (1998) AutMol Cell Biol 18, 2282-2297; and Wek, S. A., et al (1995) Mol Cell Biol 15, 4497-4506). Activated GCN2 phosphorylates its only known target, the translation initiation factor elF2a, resulting in attenuation of global protein synthesis. GCN2 also regulates Sestrin2-mediated repression of mTORC1 and induces autophagy (Talloczy, Z., et al (2002) Proc Natl Acad Sci USA 99, 190-195; Wengrod, J., et al (2015) Sci Signal 8, ra27; B'Chir, W., et al (2013) Nucleic Acids Res 41, 7683-7699; Ye, J., et al (2015) Genes Dev 29, 2331-2336; and Ravindran, R., et al (2016) Nature 531, 523-527). Together, these GCN2 effects promote the recovery of cells from amino acid shortages.

In solid tumours, GCN2 signalling is critical for cancer cell survival under conditions of nutrient deprivation (Wang, Y., et al (2013) Neoplasia 15, 989-997; Ye, J., et al (2010) EMBO J 29, 2082-2096; and Parzych, K., et al (2019) Oncogene 38, 3216-3231). GCN2 has also been shown to have a key role in MYC-driven tumour progression, by adapting protein synthesis to ensure that translation rates are compatible with the bioenergetic capacity and survival of cancer cells (Tameire, F., et al (2019) Nat Cell Biol 21, 889-899; and Schmidt, S., et al. (2019) Nat Cell Biol 21, 1413-1424). Moreover, some tumours may depend on myeloid GCN2 signals for protection from anti-cancer immune attacks (Halaby, M. J., et al (2019). Sci Immunol 4 (42), eaax8189). GCN2 depletion enhances the anti-tumour effects of asparaginase treatment (Ye, J., et al (2010) EMBO J 29, 2082-2096; and Bunpo, P., et al (2009) J Biol Chem 284, 32742-32749). Importantly, mice deficient in GCN2 do not show gross pathologies unless they receive diets that lack essential amino acids (Anthony, T. G., et al (2004) J Biol Chem 279, 36553-36561; and Zhang, P., et al (2002) Mol Cell Biol 22, 6681-6688). Taken together, these data suggest that GCN2 inhibition may be an effective cancer therapy in a diverse range of cancers.

It has also been shown that proteasome inhibitors trigger intracellular amino acid shortage, and that this effect may be the main cause of multiple myeloma cell death upon proteasome inhibitor treatment (Parzych, K., et al (2015) Cell death & disease 6, e2031; Suraweera, A., et al (2012) Mol Cell 48, 242-253; and Vabulas, R. M., and Hartl, F. U. (2005) Science 310, 1960-1963). GCN2 inhibition is therefore predicted to be particularly effective in combination with proteasome inhibitors in the treatment of multiple myeloma.

There are very few known inhibitors of GCN2. WO 2018/030466 (Takeda Pharmaceutical Company Limited) discloses a series of GCN2 inhibitor compounds having an alkynyl-phenyl core. Other GCN2 inhibitor compounds are disclosed in Fujimoto, J. et al (2019) ACS Med. Chem. Lett 10 (1), 1498-1503, and US published patent applications US 2019/0233411 and US 2019/0233425.

There is a need in the art for further GCN2 inhibitor compounds, in particular GCN2 inhibitor compounds that have high potency, and GCN2 inhibitor compounds that have good pharmacokinetic properties, such as good solubility, and therefore can be used as medicaments for the treatment of, for example, cancer.

### SUMMARY

This invention provides a compound of formula (I) or a pharmaceutically acceptable ester, amide, carbamate or salt thereof, including a pharmaceutically acceptable salt of such an ester, amide or carbamate:

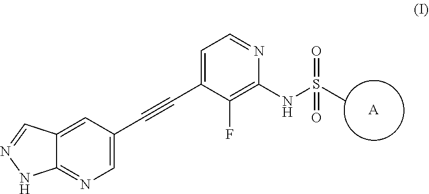

wherein A is selected from the group consisting of:

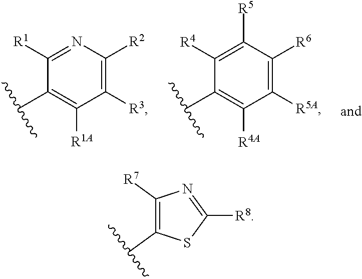

wherein R¹, R^{1A}, R² and R³ are each independently selected from the group consisting of hydrogen; halogen, cyano; C₁₋₂alkyl, optionally substituted by 1, 2 or 3 groups independently selected from the group consisting of halogen and OH; and O—C₁₋₂alkyl; and
wherein R⁴, R^{4A}, R⁵, R^{5A} and R⁶ are each independently selected from the group consisting of hydrogen; halogen, cyano; C₁₋₂alkyl, optionally substituted by 1, 2 or 3 groups independently selected from the group consisting of halogen and OH; and O—C₁₋₂alkyl; or
R⁴ and R⁵ together with the 2 atoms in the A ring to which they are attached form a 5- or 6-membered ring containing 2 oxygen atoms, in which the 2 oxygen atoms are directly attached to the 2 atoms in the A ring; and R_{1A}, R_{2A} and R₃ are each independently selected from the group consisting of hydrogen; halogen, cyano; C₁₋₂alkyl, optionally substituted by 1, 2 or 3 groups independently selected from the group consisting of halogen and OH; and O—C₁₋₂alkyl; and
wherein R⁷ and R⁸ are each independently selected from the group consisting of hydrogen; halogen, cyano; C₁₋₂alkyl, optionally substituted by 1, 2 or 3 groups independently selected from the group consisting of halogen and OH; and O—C₁₋₂alkyl;
and wherein A is not:

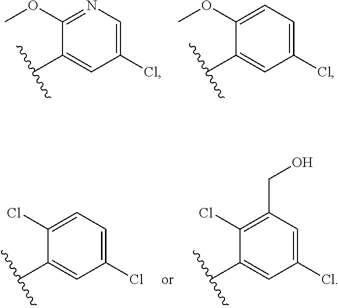

The invention also provides a pharmaceutical composition comprising a compound of formula (I) and at least one pharmaceutically acceptable carrier or excipient.

The invention further provides a pharmaceutical composition comprising a compound of formula (I), wherein said composition further comprises at least one further therapeutic agent.

The invention further provides a compound according to formula (I) or a pharmaceutical composition comprising a compound of formula (I) for use as a medicament.

The invention further provides a compound according to formula (I) or a pharmaceutical composition comprising a compound of formula (I) for use in the treatment or prophylaxis of a disease or disorder in which the inhibition of GCN2 provides a therapeutic effect.

The invention further provides a compound according to formula (I) or a pharmaceutical composition comprising a compound of formula (I) for use in the treatment of a disease or disorder selected from the group consisting of: cancer (for example solid cancers and hematological cancers).

The invention further provides a method for the treatment or prophylaxis of a disease or disorder in which the inhibition of GCN2 provides a therapeutic effect in a mammal (for example the treatment or prophylaxis of cancer in a mammal), which comprises administering to the mammal a therapeutically effective amount of a compound according to formula (I) or a pharmaceutical composition comprising a compound of formula (I).

The invention further provides the use of a compound according to formula (I) for the manufacture of a medicament for the treatment or prophylaxis of a disease or disorder in which the inhibition of GCN2 provides a therapeutic effect (for example the treatment or prophylaxis of cancer).

Further advantageous features of various embodiments of the invention are defined in the dependent claims and within the detailed description below.

### DETAILED DESCRIPTION

The invention provides compounds of formula (I) as defined above and pharmaceutical compositions comprising compounds of formula (I).

The compounds of the present invention have been found to be potent inhibitors of GCN2. Thus, the compounds of the present invention inhibit GCN2 activity and/or translation of initiation factor elF2a, resulting in attenuation of global protein synthesis in a subject.

The compounds of the invention have excellent pharmacokinetic properties. In particular, they have good solubility in aqueous media. The compounds of the invention also have good bioavailability and very suitable ‘drug-like’ pharmacokinetic properties. Therefore, the present invention also provides therapeutic uses of the compounds of formula (I) and the pharmaceutical compositions comprising compounds of formula (I).

As mentioned in the introduction, WO 2018/030466 (Takeda Pharmaceutical Company Limited) discloses a series of GCN2 inhibitor compounds having an alkynyl-phenyl core. The compounds of the current invention have been found to have surprisingly superior properties compared with the compounds disclosed in a WO 2018/030466. As demonstrated by the data herein, the compounds of the current invention are significantly more soluble in aqueous media, and they also have strong potency in the inhibition of GCN2 activity.

Furthermore, the compounds of the current invention have been found by the current inventors to have good kinase selectivity for GCN2. The kinase selectivity of compounds of the current invention has been found by the current inventors, in a KINOMEscan™ assay, to be superior to the kinase selectivity of compounds of WO 2018/030466.

The practice of the present invention employs, unless otherwise indicated, conventional techniques of organic chemistry, pharmacology, molecular biology (including recombinant techniques), cell biology, biochemistry, and immunology. Such techniques are explained in the literature, such as in “Comprehensive Organic Synthesis” (B. M. Trost & I. Fleming, eds., 1991-1992); “Handbook of Experimental Immunology” (D. M. Weir & C. C. Blackwell, eds., 1986); “Current Protocols in Molecular Biology” (F. M. Ausubel et al., eds., 1987, and periodic updates); and “Current Protocols in Immunology” (J. E. Coligan et al., eds., 1991), each of which is herein incorporated by reference in its entirety.

Various aspects of the invention are set forth below in sections; however, aspects of the invention described in one particular section are not to be limited to any particular section. Further, when a variable is not accompanied by a definition, the previous definition(s) of the variable may be applied.

### Embodiments of the Invention

The present invention provides a compound according to the general formula (I), or a pharmaceutically acceptable ester, amide, carbamate or salt thereof, including a pharmaceutically acceptable salt of such an ester, amide or carbamate:

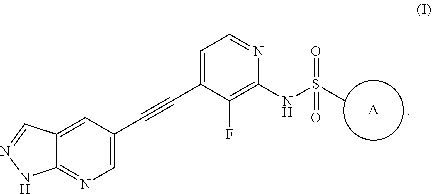

Depending upon the substituents present in the compounds of the invention, the compounds may exist as stereoisomers. In particular, the compounds of the invention may contain chiral (asymmetric) centres or the compounds as a whole may be chiral. All individual stereoisomers, as well as mixtures thereof, are included within the scope of the invention.

Diastereomeric mixtures can be separated into their individual diastereoisomers on the basis of their physical chemical differences by methods well known to those skilled in the art, such as, for example, chromatography and/or fractional crystallisation. Enantiomers can be separated by chiral HPLC column. Enantiomers can also be separated by converting the enantiomeric mixture into a diastereomeric mixture by reaction with an appropriate optically active compound (e.g. chiral auxiliary such as a chiral alcohol or Mosher's acid chloride), separating the diastereomers and converting (e.g. hydrolysing) the individual diastereomers to the corresponding pure enantiomers.

Isotopic forms, for example where a hydrogen atom is replaced with deuterium or tritium, or a carbon atom is replaced with a carbon-13 atom, are also included within the invention. Certain isotopic forms may have beneficial biological properties, for example improved metabolic stability or enhanced therapeutic activity over other isotopic forms; or a specific isotopic form may be useful for biological imaging purposes, for example, carbon-11, nitrogen-13, oxygen-15 or fluorine-18 isotopic variants may be used for positron emission tomography.

In one embodiment of the invention, A is

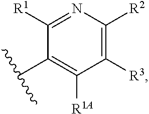

wherein R¹, R^{1A}, R² and R³ are each independently selected from the group consisting of hydrogen; halogen, cyano; C₁₋₂alkyl, optionally substituted by 1, 2 or 3 groups independently selected from the group consisting of halogen and OH; and O—C₁₋₂alkyl;
and wherein A is not:

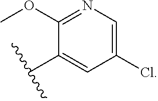 [SMILES: *c1cc(Cl)cnc1OC]

In a preferred embodiment, R¹ is selected from the group consisting of hydrogen, methyl, —CH₂CH₂OH and O—C₁₋₂alkyl. More preferably, R¹ is selected from the group consisting of methyl, —CH₂CH₂OH and O—C₁₋₂alkyl.

In a preferred embodiment, R^{1A} is hydrogen.

In a preferred embodiment, R² is selected from the group consisting of hydrogen and methyl.

In a preferred embodiment R³ is selected from the group consisting of hydrogen, fluorine, chlorine, cyano, methyl and trifluoromethyl.

In a more preferred embodiment, A is

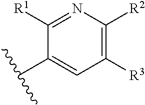

wherein
R¹ is selected from the group consisting of hydrogen, methyl, —CH₂CH₂OH and O—C₁₋₂alkyl;
R² is selected from the group consisting of hydrogen and methyl; and
R³ is selected from the group consisting of hydrogen, fluorine, chlorine, cyano, methyl and trifluoromethyl;

In an especially preferred embodiment, A is

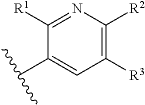

wherein
R¹ is selected from the group consisting of methyl, —CH₂CH₂OH and O—C₁₋₂alkyl;
R² is selected from the group consisting of hydrogen and methyl; and
R³ is selected from the group consisting of hydrogen, fluorine, chlorine, cyano, methyl and trifluoromethyl.

In certain preferred embodiments, the compound of the invention is a compound of the invention described in the Examples section below, or a pharmaceutically acceptable ester, amide, carbamate or salt thereof, including a pharmaceutically acceptable salt of such an ester, amide or carbamate. In particular, the compound of the invention may be a compound selected from the group consisting of:
5-Chloro-N-[3-fluoro-4-(2-{1H-pyrazolo[3,4-b]pyridin-5-yl}ethynyl)pyridin-2-yl]-2-methylpyridine-3-sulfonamide (example 4);
N-[3-Fluoro-4-(2-{1H-pyrazolo[3,4-b]pyridin-5-yl}ethynyl)pyridin-2-yl]-2-methoxy-5-(trifluoromethyl)pyridine-3-sulfonamide (example 5);
5-Chloro-N-[3-fluoro-4-(2-{1H-pyrazolo[3,4-b]pyridin-5-yl}ethynyl)pyridin-2-yl]pyridine-3-sulfonamide (example 7);
N-[3-Fluoro-4-(2-{1H-pyrazolo[3,4-b]pyridin-5-yl}ethynyl)pyridin-2-yl]-2-methoxypyridine-3-sulfonamide (example 8);
N-[3-Fluoro-4-(2-{1H-pyrazolo[3,4-b]pyridin-5-yl}ethynyl)pyridin-2-yl]-2-methoxy-5-methyl-pyridine-3-sulfonamide (example 9);
N-[3-Fluoro-4-(2-{1H-pyrazolo[3,4-b]pyridin-5-yl}ethynyl)pyridin-2-yl]-2,5-dimethylpyridine-3-sulfonamide (example 11);
5-Cyano-N-[3-fluoro-4-(2-{1H-pyrazolo[3,4-b]pyridin-5-yl}ethynyl)pyridin-2-yl]-2-methoxypyridine-3-sulfonamide (example 12);
5-Fluoro-N-[3-fluoro-4-(2-{2H-pyrazolo[4,3-b]pyridin-5-yl}ehynyl)pyridin-2-yl]-2-methoxypyridine-3-sulfonamide (example 13);
N-[3-Fluoro-4-(2-{1H-pyrazolo[3,4-b]pyridin-5-yl}ethynyl)pyridin-2-yl]-2,6-dimethylpyridine-3-sulfonamide (example 14);
5-Fluoro-N-[3-fluoro-4-(2-{2H-pyrazolo[4,3-b]pyridin-5-yl}ethynyl)pyridin-2-yl]-2-methoxypyridine-3-sulfonamide (example 15);
5-Fluoro-N-[3-fluoro-4-(2-{1H-pyrazolo[3,4-b]pyridin-5-yl}ethynyl)pyridin-2-yl]-2-methylpyridine-3-sulfonamide (example 16);
5-Chloro-2-ethoxy-N-[3-fluoro-4-(2-{1H-pyrazolo[3,4-b]pyridin-5-yl}ethynyl)-pyridin-2-yl]pyridine-3-sulfonamide (example 17);
5-Chloro-2-ethoxy-N-[3-fluoro-4-(2-{1H-pyrazolo[3,4-b]pyridin-5-yl}ethynyl)-pyridin-2-yl]pyridine-3-sulfonamide (example 18);
N-[3-Fluoro-4-(2-{1H-pyrazolo[3,4-b]pyridin-5-yl}ethynyl)pyridin-2-yl]-2,5-dimethylpyridine-3-sulfonamide (example 19); and
5-chloro-N-[3-fluoro-4-(2-{1H-pyrazolo[3,4-b]pyridin-5-yl}ethynyl)pyridin-2-yl]-2-(2-hydroxyethyl)pyridine-3-sulfonamide (example 20).

In one embodiment of the invention, A is

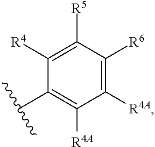

wherein R⁴, R^{4A}, R⁵, R^{5A} and R⁶ are each independently selected from the group consisting of hydrogen; halogen, cyano; C₁₋₂alkyl, optionally substituted by 1, 2 or 3 groups independently selected from the group consisting of halogen and OH; and O—C₁₋₂alkyl; or
R⁴ and R⁵ together with the 2 atoms in the A ring to which they are attached form a 5- or 6-membered ring containing 2 oxygen atoms, in which the 2 oxygen atoms are directly attached to the 2 atoms in the A ring;
and wherein A is not:

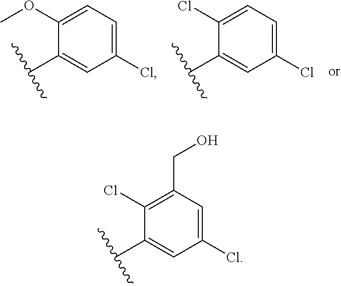

In a preferred embodiment, R^{4A} and R^{5A} are hydrogen.

In a preferred embodiment, R⁴ is selected from the group consisting of hydrogen, fluorine and —CH₂OH. More preferably, R⁴ is-CH₂OH.

In a preferred embodiment, R⁵ is selected from the group consisting of hydrogen, cyano and —OCH₃. More preferably, R⁵ is hydrogen.

In another preferred embodiment, R⁴ and R⁵ together with the 2 carbon atoms in the A ring to which they are attached form a 1,3-dioxalane ring.

In a preferred embodiment, R⁶ is selected from the group consisting of hydrogen and chlorine. More preferably, R⁶ is chlorine.

In a more preferred embodiment, A is

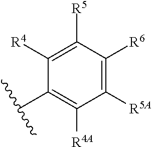

wherein:
R^{4A} and R^{5A} are hydrogen;
R⁴ is selected from the group consisting of hydrogen, fluorine and —CH₂OH;
R⁵ is selected from the group consisting of hydrogen, cyano and —OCH₃; and
R⁶ is selected from the group consisting of hydrogen and chlorine.

In another more preferred embodiment, A is

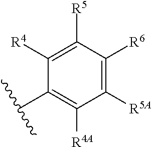

wherein:
R^{4A} and R^{5A} are hydrogen;
R⁴ and R⁵ together with the 2 carbon atoms in the A ring to which they are attached form a 1,3-dioxalane ring; and
R⁶ is selected from the group consisting of hydrogen and chlorine.

In an especially preferred embodiment, A is

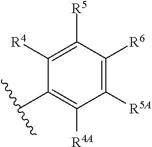

wherein:
R^{4A} and R^{5A} are hydrogen;
R⁴ is-CH₂OH; and
R⁵ is hydrogen; and
R⁶ is chlorine.

In certain preferred embodiments, the compound of the invention is a compound of the invention described in the Examples section below, or a pharmaceutically acceptable ester, amide, carbamate or salt thereof, including a pharmaceutically acceptable salt of such an ester, amide or carbamate. In particular, the compound of the invention may be a compound selected from the group consisting of:
2-Fluoro-N-[3-fluoro-4-(2-{1H-pyrazolo[3,4-b]pyridin-5-yl}ethynyl)pyridin-2-yl]-3-methoxybenzene-1-sulfonamide (example 1);
3-Cyano-N-[3-fluoro-4-(2-{1H-pyrazolo[3,4-b]pyridin-5-yl}ethynyl)pyridin-2-yl]benzene-1-sulfonamide (example 2);
N-{3-Fluoro-4-[2-(1H-indazol-5-yl)ethynyl]pyridin-2-yl}-3-methoxybenzene-1-sulfonamide (example 3); and
N-[3-Fluoro-4-(2-{1H-pyrazolo[3,4-b]pyridin-5-yl}ethynyl)pyridin-2-yl]-2H-1,3-benzodioxole-4-sulfonamide (example 10).

In one embodiment of the invention, A is

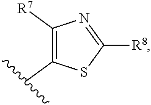

wherein R⁷ and R⁸ are each independently selected from the group consisting of hydrogen; halogen, cyano; C₁₋₂alkyl, optionally substituted by 1, 2 or 3 groups independently selected from the group consisting of halogen and OH; and O—C₁₋₂alkyl.

More preferably, R⁷ and R₈ are methyl.

In certain preferred embodiments, the compound of the invention is a compound of the invention described in the Examples section below, or a pharmaceutically acceptable ester, amide, carbamate or salt thereof, including a pharmaceutically acceptable salt of such an ester, amide or carbamate. In particular, the compound of the invention may be a compound selected from the group consisting of:
N-[3-Fluoro-4-(2-{1H-pyrazolo[3,4-b]pyridin-5-yl}ethynyl)pyridin-2-yl]-2,4-dimethyl-1,3-thiazole-5-sulfonamide (example 6).

Depending upon the substituents present in the compounds of the invention, the compounds may form esters, amides, carbamates and/or salts. Salts of compounds of the invention which are suitable for use in medicine are those wherein a counterion is pharmaceutically acceptable. Such pharmaceutically acceptable salts are described in standard texts on salt formation, see for example: P. Stahl, et al., Handbook of Pharmaceutical Salts: Properties, Selection and Use (VCHA/Wiley-VCH, 2002), or S. M. Berge, et al., “Pharmaceutical Salts”, J. Pharm. Sci., 1977, 66, 1-19. However, salts having non-pharmaceutically acceptable counterions are within the scope of the present invention, for example, for use as intermediates in the preparation of the compounds of the invention and their pharmaceutically acceptable salts, and physiologically functional derivatives. By the term “physiologically functional derivative” is meant a chemical derivative of a compound of the invention having the same physiological function as the free compound of the invention, for example, by being convertible in the body thereto. Esters, amides and carbamates are examples of physiologically functional derivatives.

Suitable salts according to the invention include those formed with organic or inorganic acids. In particular, suitable salts formed with acids according to the invention include those formed with mineral acids, strong organic carboxylic acids, such as alkanecarboxylic acids of 1 to 4 carbon atoms which are unsubstituted or substituted, for example, by halogen, such as saturated or unsaturated dicarboxylic acids, such as hydroxycarboxylic acids, such as amino acids, or with organic sulfonic acids, such as (C₁₋₄) alkyl or aryl sulfonic acids which are unsubstituted or substituted, for example by halogen. Pharmaceutically acceptable acid addition salts include those formed from hydrochloric, hydrobromic, sulphuric, nitric, citric, tartaric, acetic, phosphoric, lactic, pyruvic, acetic, trifluoroacetic, succinic, perchloric, fumaric, maleic, glycolic, lactic, salicylic, oxaloacetic, methanesulfonic, ethanesulfonic, p-toluenesulfonic, formic, benzoic, malonic, naphthalene-2-sulfonic, benzenesulfonic, isethionic, ascorbic, malic, phthalic, aspartic, and glutamic acids, lysine and arginine. Other acids such as oxalic, while not in themselves pharmaceutically acceptable, may be useful as intermediates in obtaining the compounds of the invention and their pharmaceutically acceptable acid addition salts.

Suitable salts according to the invention also include those formed with organic or inorganic bases. Pharmaceutically acceptable base salts include ammonium salts, alkali metal salts, for example those of potassium and sodium, alkaline earth metal salts, for example those of calcium and magnesium, and salts with organic bases, for example dicyclohexylamine, N-methyl-D-glucomine, morpholine, thiomorpholine, piperidine, pyrrolidine, a mono-, di- or tri-lower alkylamine, for example ethyl-, tert-butyl-, diethyl-, diisopropyl-, triethyl-, tributyl- or dimethyl-propylamine, or a mono-, di- or trihydroxy lower alkylamine, for example mono-, di- or triethanolamine.

Compounds of the invention may have an appropriate group converted to an ester, an amide or a carbamate. Typical ester and amide and carbamate groups formed from an —OH or —NHR^{G} group in the compounds of the invention include OC(O)R^{G}, NR^{G}C(O)R^{G}, NR^{G}CO₂R^{G}, OSO₂R^{G}, and NR^{G}SO₂R^{G}, where R^{G} is selected from the group consisting of C₁₋₈alkyl, C₂₋₈alkenyl, C₂₋₈alkynyl, C₃₋₈cycloalkyl and C₃₋₈cycloalkylC₁₋₈alkyl, haloC₁₋₈alkyl, dihaloC₁₋₈alkyl, trihaloC₁₋₈alkyl, phenyl and phenylC₁₋₄alkyl; more preferably R^{G} is selected from the group consisting of C₁₋₈alkyl, C₂₋₆alkenyl, C₂₋₆alkynyl, C₃₋₈cycloalkyl and C₃₋₈cycloalkylC₁₋₈alkyl.

Those skilled in the art of organic chemistry will appreciate that many organic compounds can form complexes with solvents in which they are reacted, or from which they are precipitated or crystallized. These complexes are known as “solvates”. A “pharmaceutically acceptable solvate” means a molecular complex comprising the compound of the invention and one or more pharmaceutically acceptable solvent molecules, for example, water or ethanol. For example, a complex with water is known as a “hydrate”. Solvates, such as hydrates, exist when the drug substance incorporates solvent, such as water, in the crystal lattice in either stoichiometric or non-stoichiometric amounts. Drug substances are routinely screened for the existence of hydrates since these may be encountered at any stage of the drug manufacturing process or upon storage of the drug substance or dosage form. Solvates are described in S. Byrn et al., Pharmaceutical Research, 12 (7), 1995, 954-954, and Water-Insoluble Drug Formulation, 2^{nd} edn, R. Liu, CRC Press, page 553, which are incorporated herein by reference. Accordingly, it will be understood by the skilled person that the compounds of the invention, as well as esters, amides, carbamates and/or salts thereof may therefore be present in the form of solvates, and these are also included within the scope of the present invention. Solvates of compounds of the invention, which are suitable for use in medicine, are those wherein the associated solvent is pharmaceutically acceptable. For example, as mentioned above, a hydrate is an example of a pharmaceutically acceptable solvate. However, solvates having non-pharmaceutically acceptable associated solvents may find use as intermediates in the preparation of the compounds of the invention and their pharmaceutically acceptable esters, amides, carbamates and/or salts thereof.

A compound which, upon administration to the recipient, is capable of being converted into a compound of the invention as described above, or an active metabolite or residue thereof, is known as a “prodrug”. A prodrug may, for example, be converted within the body, e.g. by hydrolysis in the blood, into its active form that has medical effects. Pharmaceutically acceptable prodrugs are described in T. Higuchi and V. Stella, Prodrugs as Novel Delivery Systems, Vol. 14 of the ACS Symposium Series (1976); “Design of Prodrugs” ed. H. Bundgaard, Elsevier, 1985; and in Edward B. Roche, ed., Bioreversible Carriers in Drug Design, American Pharmaceutical Association and Pergamon Press, 1987, which are incorporated herein by reference.

### Definitions

In the context of the present application and invention, the following definitions apply: As used herein, the term “halogen” means fluorine, chlorine, bromine, or iodine. Fluorine, chlorine or bromine are preferred. Fluorine and chlorine are particularly preferred.

As used herein, “alkyl” used alone or as a suffix or prefix, is intended to include both branched and straight chain saturated aliphatic hydrocarbon groups of the specified number of carbon atoms. For example, “C₁₋₆alkyl” denotes alkyl having 1, 2, 3, 4, 5 or 6 carbon atoms. Examples of alkyl groups include, but are not limited to, methyl, ethyl, n-propyl, i-propyl, n-butyl, i-butyl, sec-butyl, t-butyl, pentyl, and hexyl.

As used herein, the term “aryl” means phenyl or naphthyl.

As used herein, the term “cycloalkyl” means a saturated group in a ring system of the specified number of carbon atoms. For example, “C₃₋₆cycloalkyl” denotes a cycloalkyl group having 3, 4, 5 or 6 carbon atoms. A cycloalkyl group can be monocyclic, spirocyclic or bicyclic. A cycloalkyl group may have a bridge in the cyclic structure. Examples of monocyclic cycloalkyl groups include cyclopropyl, cyclobutyl and cyclopentyl. Other examples of monocyclic cycloalkyl groups are cyclohexyl, cycloheptyl and cyclooctyl.

Examples of bridged cycloalkyl groups include bicyclo[2.2.1]hept-2-yl and adamantanyl. Examples of spirocyclic cycloalkyl groups include spiro[5.5]undecanyl and spiro[5.4]decanyl.

Preferably, the cycloalkyl group is monocyclic or spirocyclic and the monocyclic or spirocyclic cycloalkyl groups may optionally be bridged.

As mentioned above, the compounds of the invention have activity as inhibitors for GCN2, and are inhibitors of GCN2. As such, the invention also provides a compound of the invention, or a composition comprising a compound of the invention, for use as a medicament, or for use in therapy. For example, the invention provides a compound of the invention, or a composition comprising a compound of the invention, together with a pharmaceutically acceptable carrier, for use as a medicament, or for use in therapy.

For the avoidance of doubt, as used herein the terms “therapy”, “treatment” and “treating” include both preventative and curative treatment of a condition, disease or disorder. It also includes slowing, interrupting, controlling or stopping the progression of a condition, disease or disorder. It also includes preventing, curing, slowing, interrupting, controlling or stopping the symptoms of a condition, disease or disorder. For example, it includes preventing the metastasis of cancer wherein the disease or disorder is cancer.

A compound of the invention, or a composition comprising a compound of the invention, may be used in the treatment of a disease or disorder in which the inhibition of GCN2 provides a therapeutic effect. As such, the compounds of the invention may be used in the treatment or prophylaxis of diseases or disorders for which inhibitors of GCN2 are indicated.

The compounds of the invention find particular application in the treatment or prophylaxis of a disease or disorder in which the inhibition of GCN2 provides a therapeutic effect, for example a disease or disorder selected from the group consisting of: cancer (for example solid cancers and hematological cancers).

The invention also provides a method of treating a subject suffering from a medical disorder or disease. The method comprises administering to the subject a therapeutically effective amount of a compound of the invention or a composition as described herein, to treat the disorder or disease. As mentioned above, a number of diseases or disorders in which the inhibition of GCN2 provides a therapeutic effect can be treated using the compounds of the invention. For example, the compounds described herein can be used to treat cancer (for example solid cancers and hematological cancers).

When a compound of the invention, or a composition comprising a compound of the invention, is used in therapy as a medicament for the treatment or prophylaxis of a disease or disorder, for example in the therapeutic uses and methods described herein, the use or method may comprise the step of administering, to a mammal, including a human, in need of such treatment or prophylaxis, a therapeutically effective amount of a compound of the invention.

The compounds of the invention find particular application in the treatment or prophylaxis of cancer. In certain embodiments, the cancer is a solid tumor or a hematological cancer (for example leukemia or multiple myeloma). In certain embodiments, the cancer is a cancer with a MYC mutation.

Examples of cancers that the compounds of the invention find particular application in the treatment or prophylaxis of include, but are not limited to: colorectal cancer (e.g., colorectal cancer, rectal cancer, anal cancer, familial colorectal cancer, hereditary nonpolyposis colorectal cancer, gastrointestinal stromal tumor), lung cancer (e.g., non-small cell lung cancer, small cell lung cancer, malignant mesothelioma), mesothelioma, pancreatic cancer (e.g., pancreatic duct cancer, pancreatic endocrine tumor), pharyngeal cancer, laryngeal cancer, esophagus cancer, gastric cancer (e.g., papillary adenocarcinoma, mucinous adenocarcinoma, adenosquamous carcinoma), duodenal cancer, small intestinal cancer, breast cancer (e.g., invasive ductal carcinoma, ductal carcinoma in situ, inflammatory breast cancer), ovarian cancer (e.g., ovarian epithelial carcinoma, extragonadal germ cell tumor, ovarian germ cell tumor, ovarian low malignant potential tumor), testis tumor, prostate cancer (e.g., hormone-dependent prostate cancer, non-hormone dependent prostate cancer, castration-resistant prostate cancer), liver cancer (e.g., hepatoma, primary liver cancer, extrahepatic bile duct cancer), thyroid cancer (e.g., medullary thyroid carcinoma), renal cancer (e.g., renal cell carcinoma (e.g., clear cell renal cell carcinoma), transitional cell carcinoma of renal pelvis and ureter), uterine cancer (e.g., cervixcancer, uterine body cancer, uterus sarcoma), gestational choriocarcinoma, brain tumor (e.g., medulloblastoma, glioma, glioblastoma, pineal astrocytoma, pilocytic astrocytoma, diffuse astrocytoma, anaplastic astrocytoma, hypophyseal adenoma), retina blastoma, skin cancer (e.g., basal cell carcinoma, malignant melanoma (melanoma)), sarcoma (e.g., rhabdomyosarcoma, leiomyosarcoma, soft tissue sarcoma, spindle cell sarcoma, osteosarcoma), malignant bone tumor, urinary bladder cancer, and hematologic cancer (e.g., multiple myeloma, smouldering myeloma, plasmacytoma, leukemia (e.g., acute myeloid leukemia, acute lymphocytic leukemia (including blast crisis of chronic leukemia)), non-Hodgkin's lymphoma, malignant lymphoma, Hodgkin's disease, chronic myeloproliferative disease), and cancer of unknown primary nucleus).

The compounds of the invention also find application as cancer growth inhibitors, cancer metastasis inhibitors, apoptosis promoters, and for the prophylaxis or treatment of precancerous lesions (e.g., bone marrow myelodysplastic syndrome, monoclonal gammopathy of undetermined significance).

In one embodiment, the compounds of the invention find particular application in the treatment or prophylaxis of osteosarcoma, acute myeloid leukemia, acute lymphocytic leukemia, multiple myeloma, pancreatic cancer, colorectal cancer, melanoma, and malignant lymphoma.

Examples of solid cancers that the compounds of the invention find particular application in the treatment or prophylaxis of include, but are not limited to: colorectal cancer (e.g., colorectal cancer, rectal cancer, anal cancer, familial colorectal cancer, hereditary nonpolyposis colorectal cancer, gastrointestinal stromal tumor), lung cancer (e.g., non-small cell lung cancer, small cell lung cancer, malignant mesothelioma), mesothelioma, pancreatic cancer (e.g., pancreatic duct cancer, pancreatic endocrine tumor), pharyngeal cancer, laryngeal cancer, esophagus cancer, gastric cancer (e.g., papillary adenocarcinoma, mucinous adenocarcinoma, adenosquamous carcinoma), duodenal cancer, small intestinal cancer, breast cancer (e.g., invasive ductal carcinoma, ductal carcinoma in situ, inflammatory breast cancer), ovarian cancer (e.g., ovarian epithelial carcinoma, extragonadal germ cell tumor, ovarian germ cell tumor, ovarian low malignant potential tumor), testis tumor, prostate cancer (e.g., hormone-dependent prostate cancer, non-hormone dependent prostate cancer, castration-resistant prostate cancer), liver cancer (e.g., hepatoma, primary liver cancer, extrahepatic bile duct cancer), thyroid cancer (e.g., medullary thyroid carcinoma), renal cancer (e.g., renal cell carcinoma (e.g., clear cell renal cell carcinoma), transitional cell carcinoma of renal pelvis and ureter), uterine cancer (e.g., cervixcancer, uterine body cancer, uterus sarcoma), gestational choriocarcinoma, brain tumor (e.g., medulloblastoma, glioma, glioblastoma, pineal astrocytoma, pilocytic astrocytoma, diffuse astrocytoma, anaplastic astrocytoma, hypophyseal adenoma), retina blastoma, skin cancer (e.g., basal cell carcinoma, malignant melanoma (melanoma)), sarcoma (e.g., rhabdomyosarcoma, leiomyosarcoma, soft tissue sarcoma, spindle cell sarcoma, osteosarcoma), malignant bone tumor, and urinary bladder cancer.

Examples of hematological cancers that the compounds of the invention find particular application in the treatment or prophylaxis of include, but are not limited to: multiple myeloma, smouldering myeloma, plasmacytoma, leukemia (e.g., acute myeloid leukemia, acute lymphocytic leukemia (including blast crisis of chronic leukemia)), non-Hodgkin's lymphoma, malignant lymphoma, Hodgkin's disease, and chronic myeloproliferative disease.

In one embodiment, the compounds of the invention find particular application in the treatment or prophylaxis of a cancer with high levels of MYC (i.e. a cancer in which the MYC gene or protein are expressed at high levels). Examples of cancers having a MYC mutation that the compounds of the invention find particular application in the treatment or prophylaxis of include, but are not limited to: prostate cancer, breast cancer (for example triple negative breast cancer), lung cancer (for example small cell lung cancer), ovarian cancer, neuroblastomas and leukemia (for example acute lymphoblastic leukemia and mixed-lineage leukemia).

The compounds of the invention also find application in conditions selected from: diabetic retinopathy, myocardial ischemia, diabetic cardiomyopathy, allergic airway inflammation, doxorubicin-induced cardiotoxicity and nonalcoholic fatty liver disease (NAFLD).

The invention also provides a method for the treatment or prophylaxis of a disease or disorder in which the inhibition of GCN2 provides a therapeutic effect in a mammal, which comprises administering to the mammal a therapeutically effective amount of a compound according to the invention, or a composition comprising a compound according to the invention. Diseases and disorders that may be treated by this method of the invention are preferably those described above.

The invention also provides the use of a compound according to the invention, for the manufacture of a medicament for the treatment or prophylaxis of a disease or disorder in which the inhibition of GCN2 provides a therapeutic effect. Diseases and disorders that may be treated by this use of the invention are preferably those described above.

The amount of active ingredient which is required to achieve a therapeutic effect will, of course, vary with the particular compound, the route of administration, the subject under treatment, including the type, species, age, weight, sex, and medical condition of the subject and the renal and hepatic function of the subject, and the particular disorder or disease being treated, as well as its severity. An ordinarily skilled physician, veterinarian or clinician can readily determine and prescribe the effective amount of the drug required to prevent, counter or arrest the progress of the condition.

Oral dosages of the present invention, when used for the indicated effects, will range between about 0.01 mg per kg of body weight per day (mg/kg/day) to about 100 mg/kg/day, preferably 0.01 mg per kg of body weight per day (mg/kg/day) to 10 mg/kg/day, and most preferably 0.1 to 5.0 mg/kg/day, for adult humans. For oral administration, the compositions are preferably provided in the form of tablets or other forms of presentation provided in discrete units containing 0.01, 0.05, 0.1, 0.5, 1.0, 2.5, 5.0, 10.0, 15.0, 25.0, 50.0, 100, and 500 milligrams of the active ingredient for the symptomatic adjustment of the dosage to the patient to be treated. A medicament typically contains from about 0.01 mg to about 500 mg of the active ingredient, preferably from about 1 mg to about 100 mg of active ingredient. Intravenously, the most preferred doses will range from about 0.1 to about 10 mg/kg/minute during a constant rate infusion. Advantageously, compounds of the invention may be administered in a single daily dose, or the total daily dosage may be administered in divided doses of two, three or four times daily. Furthermore, compounds for the invention can be administered in intranasal form via topical use of suitable intranasal vehicles, or via transdermal routes, using those forms of transdermal skin patches well known to those of ordinary skill in the art. To be administered in the form of a transdermal delivery system, the dosage administration will, of course, be continuous rather than intermittent throughout the dosage regimen.

While it is possible for the active ingredient to be administered alone, it is preferable for it to be present in a pharmaceutical formulation or composition. Accordingly, the invention provides a pharmaceutical formulation or composition comprising a compound according to the invention, and a pharmaceutically acceptable diluent, excipient or carrier (collectively referred to herein as “carrier” materials). Pharmaceutical compositions and formulations of the invention may take the form of a pharmaceutical composition or formulation as described below.

Pharmaceutical compositions according to the invention include those suitable for oral, parenteral (including subcutaneous, intradermal, intramuscular, intravenous [bolus or infusion], and intraarticular), inhalation (including fine particle dusts or mists which may be generated by means of various types of metered dose pressurized aerosols), nebulizers or insufflators, rectal, intraperitoneal and topical (including dermal, buccal, sublingual, and intraocular) administration, although the most suitable route may depend upon, for example, the condition and disorder of the recipient.

The compositions may conveniently be presented in unit dosage form and may be prepared by any of the methods well known in the art of pharmacy. All methods include the step of bringing the active ingredient into association with the carrier, which constitutes one or more accessory ingredients. In general, the compositions are prepared by uniformly and intimately bringing into association the active ingredient with liquid carriers or finely divided solid carriers or both and then, if necessary, shaping the product into the desired composition.

Compositions of the present invention suitable for oral administration may be presented as discrete units such as capsules, cachets, pills or tablets each containing a predetermined amount of the active ingredient; as a powder or granules; as a solution or a suspension in an aqueous liquid or a non-aqueous liquid, for example as elixirs, tinctures, suspensions or syrups; or as an oil-in-water liquid emulsion or a water-in-oil liquid emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Moulded tablets may be made by moulding in a suitable machine a mixture of the powdered compound moistened with an inert liquid diluent. The tablets may optionally be coated or scored and may be formulated so to provide slow or controlled release of the active ingredient therein. The compounds of the invention can, for example, be administered in a form suitable for immediate release or extended release. Immediate release or extended release can be achieved by the use of suitable pharmaceutical compositions comprising a compound of the present invention, or, particularly in the case of extended release, by the use of devices such as subcutaneous implants or osmotic pumps.

Exemplary compositions for oral administration include suspensions which can contain, for example, microcrystalline cellulose for imparting bulk, alginic acid or sodium alginate as a suspending agent, methylcellulose as a viscosity enhancer, and sweeteners or flavoring agents such as those known in the art; and immediate release tablets which can contain, for example, microcrystalline cellulose, dicalcium phosphate, starch, magnesium stearate, calcium sulfate, sorbitol, glucose and/or lactose and/or other excipients, binders, extenders, disintegrants, diluents and lubricants such as those known in the art. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Disintegrators include without limitation starch, methylcellulose, agar, bentonite, xanthan gum and the like. The compounds of the invention can also be delivered through the oral cavity by sublingual and/or buccal administration. Molded tablets, compressed tablets or freeze-dried tablets are exemplary forms which may be used. Exemplary compositions include those formulating the present compound(s) with fast dissolving diluents such as mannitol, lactose, sucrose and/or cyclodextrins. Also included in such compositions may be high molecular weight excipients such as celluloses (avicel) or polyethylene glycols (PEG). Such compositions can also include an excipient to aid mucosal adhesion such as hydroxypropyl cellulose (HPC), hydroxypropyl methylcellulose (HPMC), sodium carboxymethyl cellulose (SCMC), maleic anhydride copolymer (e.g. Gantrez), and agents to control release such as polyacrylic copolymer (e.g. Carbopol 934). Lubricants, glidants, flavors, coloring agents and stabilizers may also be added for ease of fabrication and use. Lubricants used in these dosage forms include sodium oleate, sodium stearate, magnesium stearate, sodium benzoate, sodium acetate, sodium chloride and the like. For oral administration in liquid form, the oral drug components can be combined with any oral, non-toxic, pharmaceutically acceptable inert carrier such as ethanol, glycerol, water, and the like.

The compounds of the present invention can also be administered in the form of liposome delivery systems, such as small unilamellar vesicles, large unilamellar vesicles and multilamellar vesicles. Liposomes can be formed from a variety of phospholipids, 1,2-dipalmitoylphosphatidylcholine, phosphatidyl ethanolamine (cephaline), or phosphatidylcholine (lecithin).

Compositions for parenteral administration include aqueous and non-aqueous sterile injection solutions which may contain anti-oxidants, buffers, bacteriostats and solutes which render the composition isotonic with the blood of the intended recipient; and aqueous and non-aqueous sterile suspensions which may include suspending agents and thickening agents. The compositions may be presented in unit-dose or multi-dose containers, for example sealed ampoules and vials, and may be stored in a freeze-dried (lyophilised) condition requiring only the addition of the sterile liquid carrier, for example saline or water-for-injection, immediately prior to use. Extemporaneous injection solutions and suspensions may be prepared from sterile powders, granules and tablets of the kind previously described. Exemplary compositions for parenteral administration include injectable solutions or suspensions which can contain, for example, suitable non-toxic, parenterally acceptable diluents or solvents, such as 1,3-butanediol, water, Ringer's solution, an isotonic sodium chloride solution, or other suitable dispersing or wetting and suspending agents, including synthetic mono- or diglycerides, and fatty acids, including oleic acid, or Cremaphor®.

Exemplary compositions for nasal, aerosol or inhalation administration include solutions in saline, which can contain, for example, benzyl alcohol or other suitable preservatives, absorption promoters to enhance bioavailability, and/or other solubilizing or dispersing agents such as those known in the art.

Compositions for rectal administration may be presented as a suppository with the usual carriers such as cocoa butter, synthetic glyceride esters or polyethylene glycol. Such carriers are typically solid at ordinary temperatures but liquefy and/or dissolve in the rectal cavity to release the drug.

Compositions for topical administration in the mouth, for example buccally or sublingually, include lozenges comprising the active ingredient in a flavoured basis such as sucrose and acacia or tragacanth, and pastilles comprising the active ingredient in a basis such as gelatin and glycerine or sucrose and acacia. Exemplary compositions for topical administration include a topical carrier such as Plastibase® (mineral oil gelled with polyethylene).

Preferred unit dosage compositions are those containing an effective dose, as hereinbefore recited, or an appropriate fraction thereof, of the active ingredient.

It should be understood that in addition to the ingredients particularly mentioned above, the compositions of this invention may include other agents conventional in the art having regard to the type of composition in question, for example, those suitable for oral administration may include flavouring agents.

Whilst a compound of the invention may be used as the sole active ingredient in a medicament, it is also possible for the compound to be used in combination with one or more further therapeutic agents. Thus, the invention also provides a compound according to the invention together with a further therapeutic agent, for simultaneous, sequential or separate administration. Such further therapeutic agents may be further compounds according to the invention, or they may be different therapeutic agents, for example another GCN2 inhibitor. The further therapeutic agent may also be a therapeutic agent for use in the treatment or prophylaxis of a disease or disorder in which the inhibition of GCN2 provides a therapeutic effect, for example a disease or disorder selected from the group consisting of cancer (for example solid cancers and hematological cancers), and austoimmune diseases, and in particular cancer.

Therefore, in one embodiment, the further therapeutic agent may be a different therapeutic agent for use in the treatment or prophylaxis of cancer, for example it may be a chemotherapeutic agent selected from the group consisting of L-asparaginase (ASNase), a proteasome inhibitor (for example bortezomib, carfilzomib, ixazomib, or marizomib), immunomodulatory drugs (for example, thalidomide, lenalidomide and pomalidomide), SINE compounds (for example selinexor), monocolonal antibodies (for example, such as rituximab, daratumumab, isatuximab, herceptin and avastin), alkylating agents, alkyl sulfonates, aziridines, ethylenimines and methylamelamines, acetogenins, a camptothecin, bryostatin, callystatin, CC-1065, cryptophycins, dolastatin, duocarmycin, eleutherobin, pancratistatin, a sarcodictyin, spongistatin, nitrogen mustards, antibiotics, enediyne antibiotics, dynemicin, bisphosphonates, esperamicin, chromoprotein enediyne antibiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin, antimetabolites, erlotinib, vemurafenib, crizotinib, sorafenib, ibrutinib, enzalutamide, folic acid analogues, purine analogs, androgens, anti-adrenals, folic acid replenisher such as folinic acid, aceglatone, aldophosphamide glycoside, aminolevulinic acid, eniluracil, amsacrine, bestrabucil, bisantrene, edatraxate, defofamine, demecolcine, diaziquone, elfornithine, elliptinium acetate, an epothilone, etoglucid, gallium nitrate, hydroxyurea, lentinan, lonidainine, maytansinoids, mitoguazone, mitoxantrone, mopidanmol, nitraerine, pentostatin, phenamet, pirarubicin, losoxantrone, podophyllinic acid 2-ethylhydrazide, procarbazine, PSK® polysaccharide complex (JHS Natural Products, Eugene, OR), razoxane, rhizoxin, sizofiran, spirogermanium, tenuazonic acid, triaziquone; 2,2′,2″-trichlorotriethylamine, trichothecenes (especially T-2 toxin, verracurin A, roridin A and anguidine), urethan, vindesine, dacarbazine, mannomustine, mitobronitol, mitolactol, pipobroman, gacytosine, arabinoside (“Ara-C”), cyclophosphamide, thiotepa, taxoids, chloranbucil, gemcitabine, 6-thioguanine, mercaptopurine, methotrexate, platinum analogs, vinblastine, platinum, etoposide (VP-16), ifosfamide, mitoxantrone, vincristine, vinorelbine, novantrone, teniposide, edatrexate, daunomycin, aminopterin, xeloda, ibandronate, irinotecan (Camptosar, CPT-11), topoisomerase inhibitor RFS 2000, difluorometlhylornithine, asparaginase, retinoids, capecitabine, combretastatin, leucovorin, oxaliplatin, inhibitors of PKC-alpha, Raf, H-Ras, EGFR and VEGF-A that reduce cell proliferation, and pharmaceutically acceptable salts, acids or derivatives thereof, and combinations thereof.

In another embodiment, the further therapeutic agent may be a checkpoint inhibitor, for example an agent or antibody that inhibits one or more of CTLA4, PD-1, PD-L1, LAG-3, B7-H3, B7-H4, TIM3, VISTA and KIR.

In certain embodiments the compound of the invention is administered in combination with L-asparaginase (ASNase). Such a combination treatment may be used for the treatment of cancer, and in particular for the treatment of an acute lymphocytic leukemia (including blast crisis of chronic leukemia) and non-Hodgkin's lymphoma. Such a combination treatment may also be used for the treatment of cancer tumor resistant or tolerant to asparaginase, for example a cancer selected from the group consisting of acute lymphocytic leukemia (including blast crisis of chronic leukemia) and non-Hodgkin's lymphoma.

In certain embodiments the compound of the invention is administered in combination with a proteasome inhibitor, for example bortezomib, carfilizomib, ixazomib, marozomib or oprozomib. Such a combination treatment may be used for the treatment of cancer, and in particular for the treatment of a hematological cancer, for example Hodgkin's lymphoma, multiple myeloma, smouldering myeloma, and the premalignant condition, monoclonal gammopathy of undetermined significance.

In embodiments where the compounds of the invention are used in combination with other agent(s) for use in the treatment or prophylaxis of a disease or disorder in which the inhibition of GCN2 provides a therapeutic effect, the individual components of such combinations can be administered separately at different times during the course of therapy or concurrently in divided or single combination forms. The present invention is therefore to be understood as embracing all such regimes of simultaneous or alternating treatment and the term “administering” is to be interpreted accordingly. It will be understood that the scope of combinations of the compounds of this invention with other agents for use in the treatment or prophylaxis of a disease or disorder in which the inhibition of GCN2 provides a therapeutic effect includes in principle any combination with any pharmaceutical composition useful for treating a disease or disorder in which the inhibition of GCN2 provides a therapeutic effect.

The above other therapeutic agents, when employed in combination with the compounds of the present invention, may be used, for example, in those amounts indicated in the Physicians' Desk Reference (PDR) or as otherwise determined by one of ordinary skill in the art.

The compounds of the invention as described above also find use in combination with radiation therapy for the treatment of cancer.

Furthermore, the compound of the present invention may be used in combination with a non-drug therapy. Specifically, the compound of the present invention or the combination agent of the present invention can be used in combination with, for example, a non-drug therapy such as (1) operation, (2) hypertensive chemical therapy using angiotensin II and the like, 30 (3) gene therapy, (4) hyperthermic therapy, (5) cryotherapy; (6) laser ablation method; (7) radiation therapy; (8) diet therapy (e.g., amino acid restriction diet) and the like.

For example, using the compound of the present invention or the combination agent of the present invention before or after the aforementioned surgery and the like, or before or after the treatment of two or three kinds of these in combination, effects such as inhibition of expression of resistance, prolongation of disease-free survival, suppression of cancer metastasis or recurrence, prolongation of life and the like can be achieved.

In addition, the treatment with the compound of the present invention or the combination agent of the present invention can be combined with a supporting therapy, for example (i) administration of antibiotics (for example, P-lactam system such as pansporin and the like, macrolide system such as clarithromycin and the like) for complications of various infectious diseases, (ii) administration of intravenous hyperalimentation, amino acid preparation, multiple vitamin preparation for improving malnutrition, (iii) morphine administration for pain relief, (iv) administration of medicament for improving side effects such as nausea, vomiting, anorexia, diarrhea, leucopenia, thrombocytopenia, hemoglobin concentration reduction, hair loss, hepatopathy, renopathy, DIC, fever and the like and (v) administration of medicament for suppressing multiple drug resistance of cancer and the like.

The compounds of the invention as described above also find use, optionally in labelled form, as a diagnostic agent for the diagnosis of conditions associated with a disease or disorder in which the inhibition of GCN2 provides a therapeutic effect. For example, such a compound may be radioactively labelled.

In addition to their use in therapeutic medicine, compounds according to the invention may also be useful as pharmacological tools in the development and standardization of in vitro and in vivo test systems for the evaluation of other compounds with similar activity. Furthermore, compounds of the invention may be used as molecular probes to identify and/or locate the target of their action, such as a target within the airways, as well as employed as a diagnostic tool for diagnosis of a disease or condition in vivo, ex vivo or in vitro, or as synthetic precursors to such probes. Molecular probes of the invention may include reactive, labeled (i.e. compounds of the invention wherein one or several of the composing atoms have been enriched with a radioactive or by other means detectable isotope), and fluorescent compounds as well known to the one skilled in the art.

The following Examples illustrate the invention.

### List of Abbreviations

aq.—aqueous
anh.—anhydrous
ACN—acetonitrile
CDCl₃—deuterated chloroform
DCM—dichloromethane
DIPEA—N,N-diisopropylethylamine
DMAP—4-—dimethylaminopyridine
DMSO—dimethylsulfoxide
DMSO—d₆-deuterated dimethylsulfoxide
EA—ethyl acetate
eq.—equivalent
FC—flash chromatography
¹H NMR—proton nuclear magnetic resonance
HPLC—high performance liquid chromatography
i-PrOH—isopropanol
MeOH—methanol
MS—mass spectrometry
r.t.—room temperature
RT—retention time
sat.—saturated
sol.—solution
TEA—triethylamine
THF—tetrahydrofuran
Y—yield

### Analytical Methods Description:

All ¹H NMR spectra were measured on Bruker Avance III HD 400 MHz or Bruker Fourier 300 MHz NMR spectrometer.

### LCMS (Method A)

Apparatus: Dionex UHPLC Ultimate 3000 with DAD detector/Thermo Scientific MSQ Plus Column: Kinetex® 2.6 μm XB-C18 (4.6×50 mm), 110A, column no. 00B-4496-E0. Internal column no. 019
Reagents:—Formic acid≥98%, Sigma-Aldrich
   Acetonitrile for HPLC UV/gradient grade, Baker
   μQ-water for LCMS
HPLC conditions:—Wavelength range: (190-340) nm±4 nm
   Flow: 1.0 ml/min
   Column temperature: 25° C.
   Autosampler temperature: 20° C.
   Analysis time: 6 min
   Elution: gradient

| Time [min] | Mobile phase A [%] | Mobile phase B [%] | Flow [ml/min] |
|---|---|---|---|
| 0.0 | 70 | 30 | 1.0 |
| 3.35 | 20 | 80 | 1.0 |
| 3.75 | 20 | 80 | 1.0 |
| 3.90 | 5 | 95 | 1.0 |
| 4.75 | 5 | 95 | 1.0 |
| 5.00 | 70 | 30 | 1.0 |
| 6.00 | 70 | 30 | 1.0 |

Mobile phase A: 0.1% v/v water solution of formic acid
Mobile phase B: 0.1% v/v acetonitrile solution of formic acid
Solution for syringe washing: 20% MeOH
MS conditions:—Mass range: 100-1000 m/z
   Ionization: alternate
   Scan speed: 12 000 amu/sec

### LCMS (Method B):

Apparatus: Dionex UHPLC Ultimate 3000 with DAD detector/Thermo Scientific ISQ EC-Mass Spectrometer
Column: Kinetex® 2.6 μm XB-C18 (4.6×50 mm), 110A, column no. 00B-4496-E0. Internal column no. 036
Reagents:—Formic acid≥98%, Sigma-Aldrich
   Acetonitrile for HPLC UV/gradient grade, Baker
   μQ-water for LCMS
HPLC conditions: Wavelength range: (190-340) nm±4 nm
   Flow: 1.0 ml/min
   Column temperature: 25° C.
   Autosampler temperature: 20° C.
   Injection volume: 2.0 μl
   Analysis time: 6 min
   Elution: gradient

| Time [min] | Mobile phase A [%] | Mobile phase B [%] | Flow [ml/min] |
|---|---|---|---|
| 0.0 | 70 | 30 | 1.0 |
| 3.35 | 20 | 80 | 1.0 |
| 3.75 | 20 | 80 | 1.0 |
| 3.90 | 5 | 95 | 1.0 |
| 4.75 | 5 | 95 | 1.0 |
| 5.00 | 70 | 30 | 1.0 |
| 6.00 | 70 | 30 | 1.0 |

Mobile phase A: 0.1% v/v water solution of formic acid
Mobile phase B: 0.1% v/v acetonitrile solution of formic acid
Solution for syringe washing: 20% MeOH
MS conditions:—Mass range: 100-1000 m/z
   Ionization: alternate
   Scan speed: 12 000 amu/sec

### LCMS (Method C):

Apparatus: Dionex UHPLC Ultimate 3000 with DAD detector/Thermo Scientific ISQ EC-Mass Spectrometer
Column: Kinetex® 2.6 μm XB-C18 (4.6×50 mm), 110A, column no. 00B-4496-E0. Internal column no. 36
Reagents:—Formic acid≥98%, Sigma-Aldrich
   Acetonitrile for HPLC UV/gradient grade, Baker
   μQ-water for LCMS
   HPLC conditions:—Wavelength range: (190-340) nm±4 nm
   Flow: 1.0 ml/min
   Column temperature: 25° C.
   Autosampler temperature: 20° C.
   Injection volume: 2.0 μl
   Analysis time: 6 min
   Elution: gradient

| Time [min] | Mobile phase A [%] | Mobile phase B [%] | Flow [ml/min] |
|---|---|---|---|
| 0.0 | 80 | 20 | 1.0 |
| 3.35 | 40 | 60 | 1.0 |
| 3.75 | 40 | 60 | 1.0 |
| 3.90 | 5 | 95 | 1.0 |
| 4.75 | 5 | 95 | 1.0 |
| 5.00 | 80 | 20 | 1.0 |
| 6.00 | 80 | 20 | 1.0 |

Mobile phase A: 0.1% v/v water solution of formic acid
Mobile phase B: 0.1% v/v acetonitrile solution of formic acid
Solution for syringe washing: 20% MeOH
MS conditions:—Mass range: 100-1000 m/z
   Ionization: alternate
   Scan speed: 12 000 amu/sec

### LCMS (Method D):

Apparatus: Dionex UHPLC Ultimate 3000 with DAD detector/Thermo Scientific ISQ EC-Mass Spectrometer
Column: Kinetex® 2.6 μm XB-C18 (4.6×50 mm), 110A, column no. 00B-4496-E0. Internal column no. 036
Reagents:—Formic acid≥98%, Sigma-Aldrich
   Acetonitrile for HPLC UV/gradient grade, Baker
   μQ-water for LCMS
HPLC conditions: Wavelength range: (190-340) nm±4 nm
   Flow: 1.0 ml/min
   Column temperature: 25° C.
   Autosampler temperature: 20° C.
   Injection volume: 2.0 μl
   Analysis time: 6 min
   Elution: gradient

| Time [min] | Mobile phase A [%] | Mobile phase B [%] | Flow [ml/min] |
|---|---|---|---|
| 0.0 | 80 | 20 | 1.0 |
| 3.35 | 20 | 80 | 1.0 |
| 3.75 | 20 | 80 | 1.0 |
| 3.90 | 5 | 95 | 1.0 |
| 4.75 | 5 | 95 | 1.0 |
| 5.00 | 80 | 20 | 1.0 |
| 6.00 | 80 | 20 | 1.0 |

Mobile phase A: 0.1% v/v water solution of formic acid
Mobile phase B: 0.1% v/v acetonitrile solution of formic acid
Solution for syringe washing: 20% MeOH
MS conditions:—Mass range: 100-1000 m/z
   Ionization: alternate
   Scan speed: 12 000 amu/sec

### LCMS (Method E)

Apparatus: Dionex UHPLC Ultimate 3000 with DAD detector/Thermo Scientific MSQ Plus
Column: Kinetex® 2.6 μm XB-C18 (4.6×50 mm), 110A, column no. 00B-4496-E0, internal column no. 019
Reagents:—Formic acid≥98%, Sigma-Aldrich
   Acetonitrile for HPLC UV/gradient grade, Baker
   μQ-water for LCMS
HPLC conditions: Wavelength range: (190-340) nm±4 nm
   Flow: 1.0 ml/min
   Column temperature: 25° C.
   Autosampler temperature: 20° C.
   Analysis time: 6 min
   Elution: gradient

| Time [min] | Mobile phase A [%] | Mobile phase B [%] | Flow [ml/min] |
|---|---|---|---|
| 0.0 | 80 | 20 | 1.0 |
| 3.35 | 20 | 80 | 1.0 |
| 3.75 | 20 | 80 | 1.0 |
| 3.90 | 5 | 95 | 1.0 |
| 4.75 | 5 | 95 | 1.0 |
| 5.00 | 80 | 20 | 1.0 |
| 6.00 | 80 | 20 | 1.0 |

Mobile phase A: 0.1% v/v water solution of formic acid
Mobile phase B: 0.1% v/v acetonitrile solution of formic acid
Solution for syringe washing: 20% MeOH
MS conditions:—Mass range: 100-1000 m/z
   Ionization: alternate
   Scan speed: 12 000 amu/sec

### LCMS (Method F)

Apparatus: Dionex UHPLC Ultimate 3000 with DAD detector/Thermo Scientific MSQ Plus
Column: Kinetex® 2.6 μm XB-C18 (4.6×50 mm), 110A, column no. 00B-4496-E0, internal column no. 019
Reagents:—Formic acid≥98%, Sigma-Aldrich
   Acetonitrile for HPLC UV/gradient grade, Baker
   μQ-water for LCMS
HPLC conditions: Wavelength range: (190-350) nm±4 nm
   Flow: 1.0 ml/min
   Column temperature: 25° C.
   Autosampler temperature: 20° C.
   Analysis time: 7 min
   Elution: gradient

| Time [min] | Mobile phase A [%] | Mobile phase B [%] | Flow [ml/min] |
|---|---|---|---|
| 0.0 | 95 | 5 | 1.0 |
| 1.0 | 95 | 5 | 1.0 |
| 4.75 | 20 | 80 | 1.0 |
| 5.25 | 20 | 80 | 1.0 |
| 6.0 | 95 | 5 | 1.0 |
| 7.0 | 95 | 5 | 1.0 |

Mobile phase A: 0.1% v/v water solution of formic acid
Mobile phase B: 0.1% v/v acetonitrile solution of formic acid
Solution for syringe washing: 20% MeOH
MS conditions:—Mass range: 100-1000 m/z
   Ionization: alternate
   Scan speed: 12 000 amu/sec

### LCMS (Method G)

Apparatus: Dionex UHPLC Ultimate 3000 with DAD detector/Thermo Scientific ISQ EC-Mass Spectrometer
Column: Kinetex® 2.6 μm XB-C18 (4.6×50 mm), 110A, column no. 00B-4496-E0, internal column no. 036
Reagents:—Formic acid≥98%, Sigma-Aldrich
   Acetonitrile for HPLC UV/gradient grade, Baker
   μQ-water for LCMS
HPLC conditions: Wavelength range: (190-350) nm±4 nm
   Flow: 1.0 ml/min
   Column temperature: 25° C.
   Autosampler temperature: 20° C.
   Analysis time: 6 min
   Elution: gradient

| Time [min] | Mobile phase A [%] | Mobile phase B [%] | Flow [ml/min] |
|---|---|---|---|
| 0.00 | 70 | 30 | 1.0 |
| 3.35 | 50 | 50 | 1.0 |
| 3.75 | 50 | 50 | 1.0 |
| 3.90 | 5 | 95 | 1.0 |
| 4.75 | 5 | 95 | 1.0 |
| 5.00 | 70 | 30 | 1.0 |
| 6.00 | 70 | 30 | 1.0 |

Mobile phase A: 0.1% v/v water solution of formic acid
Mobile phase B: 0.1% v/v acetonitrile solution of formic acid
Solution for syringe washing: 20% MeOH
MS conditions:—Mass range: 100-1000 m/z
   Ionization: alternate
   Scan speed: 12 000 amu/sec

### List of Example Names

Example 1:2-Fluoro-N-[3-fluoro-4-(2-{1H-pyrazolo[3,4-b]pyridin-5-yl}ethynyl)pyridin-2-yl]-3-methoxybenzene-1-sulfonamide
Example 2:3-Cyano-N-[3-fluoro-4-(2-{1H-pyrazolo[3,4-b]pyridin-5-yl}ethynyl)pyridin-2-yl]benzene-1-sulfonamide
Example 3: N-{3-Fluoro-4-[2-(1H-indazol-5-yl)ethynyl]pyridin-2-yl}-3-methoxybenzene-1-sulfonamide
Example 4:5-Chloro-N-[3-fluoro-4-(2-{1H-pyrazolo[3,4-b]pyridin-5-yl}ethynyl)pyridin-2-yl]-2-methylpyridine-3-sulfonamide
Example 5: N-[3-Fluoro-4-(2-{1H-pyrazolo[3,4-b]pyridin-5-yl}ethynyl)pyridin-2-yl]-2-methoxy-5-(trifluoromethyl)pyridine-3-sulfonamide
Example 6: N-[3-Fluoro-4-(2-{1H-pyrazolo[3,4-b]pyridin-5-yl}ethynyl)pyridin-2-yl]-2,4-dimethyl-1,3-thiazole-5-sulfonamide
Example 7:5-Chloro-N-[3-fluoro-4-(2-{1H-pyrazolo[3,4-b]pyridin-5-yl}ethynyl)pyridin-2-yl]pyridine-3-sulfonamide
Example 8: N-[3-Fluoro-4-(2-{1H-pyrazolo[3,4-b]pyridin-5-yl}ethynyl)pyridin-2-yl]-2-methoxypyridine-3-sulfonamide
Example 9: N-[3-Fluoro-4-(2-{1H-pyrazolo[3,4-b]pyridin-5-yl}ethynyl)pyridin-2-yl]-2-methoxy-5-methyl-pyridine-3-sulfonamide
Example 10: N-[3-Fluoro-4-(2-{1H-pyrazolo[3,4-b]pyridin-5-yl}ethynyl)pyridin-2-yl]-2H-1,3-benzodioxole-4-sulfonamide
Example 11: N-[3-Fluoro-4-(2-{1H-pyrazolo[3,4-b]pyridin-5-yl}ethynyl)pyridin-2-yl]-2,5-dimethylpyridine-3-sulfonamide
Example 12:5-Cyano-N-[3-fluoro-4-(2-{1H-pyrazolo[3,4-b]pyridin-5-yl}ethynyl)pyridin-2-yl]-2-methoxypyridine-3-sulfonamide
Example 13:5-Fluoro-N-[3-fluoro-4-(2-{2H-pyrazolo[4,3-b]pyridin-5-yl}ehynyl)pyridin-2-yl]-2-methoxypyridine-3-sulfonamide
Example 14: N-[3-Fluoro-4-(2-{1H-pyrazolo[3,4-b]pyridin-5-yl}ethynyl)pyridin-2-yl]-2,6-dimethylpyridine-3-sulfonamide
Example 15:5-Fluoro-N-[3-fluoro-4-(2-{2H-pyrazolo[4,3-b]pyridin-5-yl}ethynyl)pyridin-2-yl]-2-methoxypyridine-3-sulfonamide
Example 16:5-Fluoro-N-[3-fluoro-4-(2-{1H-pyrazolo[3,4-b]pyridin-5-yl}ethynyl)pyridin-2-yl]-2-methylpyridine-3-sulfonamide
Example 17:5-Chloro-2-ethoxy-N-[3-fluoro-4-(2-{1H-pyrazolo[3,4-b]pyridin-5-yl}ethynyl)-pyridin-2-yl]pyridine-3-sulfonamide
Example 18:5-Chloro-2-ethoxy-N-[3-fluoro-4-(2-{1H-pyrazolo[3,4-b]pyridin-5-yl}ethynyl)-pyridin-2-yl]pyridine-3-sulfonamide
Example 19: N-[3-Fluoro-4-(2-{1H-pyrazolo[3,4-b]pyridin-5-yl}ethynyl)pyridin-2-yl]-2,5-dimethylpyridine-3-sulfonamide
Example 20:5-chloro-N-[3-fluoro-4-(2-{1H-pyrazolo[3,4-b]pyridin-5-yl}ethynyl)pyridin-2-yl]-2-(2-hydroxyethyl)pyridine-3-sulfonamide

### Example 1:2-Fluoro-N-[3-fluoro-4-(2-{1H-pyrazolo[3,4-b]pyridin-5-yl}ethynyl)pyridin-2-yl]-3-methoxybenzene-1-sulfonamide

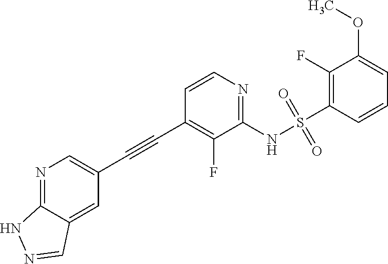 [SMILES: COc1cccc(S(=O)(=O)Nc2nccc(C#Cc3cnc4[nH]ncc4c3)c2F)c1F]

### Step 1:2-Fluoro-N-(3-fluoro-4-iodopyridin-2-yl)-3-methoxybenzene-1-sulfonamide

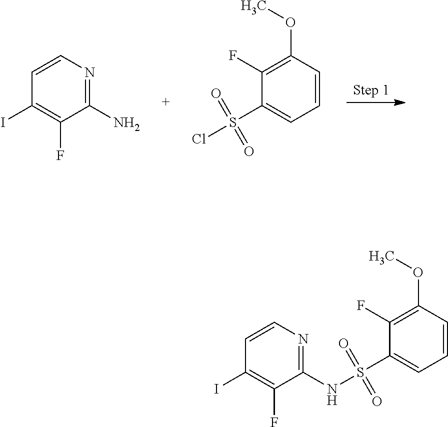 [SMILES: Nc1nccc(I)c1F ; COc1cccc(S(=O)(=O)Cl)c1F ; COc1cccc(S(=O)(=O)Nc2nccc(I)c2F)c1F]

To the solution of 3-fluoro-4-iodopyridin-2-amine (100 mg, 0.42 mmol, 1.0 eq.) in anh. THF (3 ml), pre-cooled to 0° C., NaH (60% in oil, 67 mg, 1.681 mmol, 4.0 eq.) was added and the mixture was stirred at the same temperature for 1 h. Next, 2-fluoro-3-methoxybenzene-1-sulfonyl chloride (94 mg, 0.42 mmol, 1.0 eq.), dissolved in anh. THF (1 ml), was added dropwise. The reaction mixture was allowed to warm to r.t. while stirring overnight. After that time, reaction mixture was quenched with NH₄Cl aq. sat. sol. (2 ml), EA (5 ml) was added and the layers were separated. The aqueous layer was additionally extracted with EA (2×5 ml). Organic layers were combined, dried over sodium sulphate, filtered and evaporated to dryness to give crude 2-fluoro-N-(3-fluoro-4-iodopyridin-2-yl)-3-methoxybenzene-1-sulfonamide (100 mg, 79% purity, Y: 44%) as an orange solid, which was used for the next step without purification.

MS m/z: [M+H]⁺ 427.00

### Step 2: N-{4-[2-(2-Aminopyrimidin-5-yl)ethynyl]-3-chloropyridin-2-yl}-5-chloro-2-methoxypyridine-3-sulfonamide

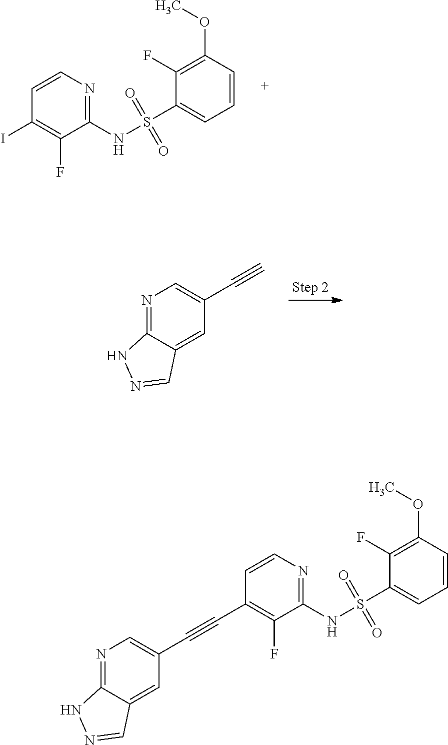 [SMILES: COc1cccc(S(=O)(=O)Nc2nccc(I)c2F)c1F ; C#Cc1cnc2[nH]ncc2c1 ; COc1cccc(S(=O)(=O)Nc2nccc(C#Cc3cnc4[nH]ncc4c3)c2F)c1F]

2-Fluoro-N-(3-fluoro-4-iodopyridin-2-yl)-3-methoxybenzene-1-sulfonamide (100 mg, 79% purity, 0.185 mmol, 1.0 eq.), 5-ethynyl-1H-pyrazolo[3,4-b]pyridine (27 mg, 0.185 mmol, 1.0 eq.) and TEA (0.14 ml, 1.020 mmol, 5.5 eq.) were dissolved in anh. DMF (5 ml) and the mixture was degassed with argon for 15 minutes. Next, copper (I) iodide (7 mg, 0.037 mmol, 0.2 eq.) and bis(triphenylphosphine) palladium (II) chloride (13 mg, 0.019 mmol, 0.1 eq.) were added and the mixture was stirred in a microwave for 40 minutes at 120° C. The reaction mixture was evaporated to dryness and MeOH was added. Precipitated brown solid was filtered and dried. The crude was purified using prep-HPLC to give 2-fluoro-N-[3-fluoro-4-(2-{1H-pyrazolo[3,4-b]pyridin-5-yl}ethynyl)pyridin-2-yl]-3-methoxybenzene-1-sulfonamide (10 mg, Y: 12%) as a yellow solid.

¹H NMR (400 MHZ, DMSO-d₆) δ 14.02 (s, 1H), 11.76 (s, 1H), 8.74 (d, J=2.0 Hz, 1H), 8.60 (d, J=2.0 Hz, 1H), 8.25 (d, J=1.4 Hz, 1H), 7.97 (s, 1H), 7.45 (d, J=6.9 Hz, 2H), 7.32 (s, 1H), 3.88 (s, 3H).

LCMS (Method A) RT: 2.083 min

MS m/z: [M+H]⁺ 441.98

Examples 2-20 were synthesized using methods analogous to those described above:

| | | ¹H NMR (400 MHz, | |
|---|---|---|---|
| Example | Structure | DMSO-d₆) δ | LCMS |
| 2 | 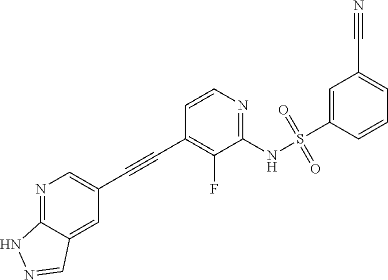 [SMILES: N#Cc1cccc(S(=O)(=O)Nc2nccc(C#Cc3cnc4[nH]ncc4c3)c2F)c1] | 14.01 (s, 1H), 11.72 (s, 1H), 8.73 (d, J = 2.0 Hz, 1H), 8.59 (d, J = 2.0 Hz, 1H), 8.37 (s, 1H), 8.31-8.22 (m, 2H), 8.13 (d, J = 7.9 Hz, 1H), 8.01 (s, 1H), 7.82 (t, J = 7.9 Hz, 1H), 7.26 (s, 1H) | Method B RT: 2.073 min m/z [M − H]⁺ 419.12 |
| 3 | 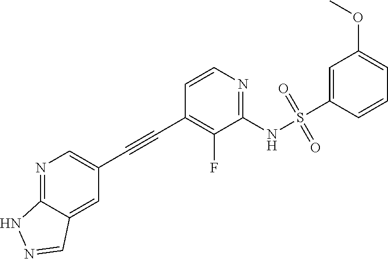 [SMILES: COc1cccc(S(=O)(=O)Nc2nccc(C#Cc3cnc4[nH]ncc4c3)c2F)c1] | 14.03 (s, 1H), 11.46 (s, 1H), 8.73 (d, J = 2.0 Hz, 1H), 8.59 (d, J = 2.0 Hz, 1H), 8.25 (s, 1H), 8.00 (d, J = 42.1 Hz, 1H), 7.52 (d, J = 6.8 Hz, 3H), 7.21 (d, J = 7.5 Hz, 2H), 3.83 (s, 3H). | Method B RT: 2.197 min m/z [M − H]⁺ 424.08 |
| 4 | 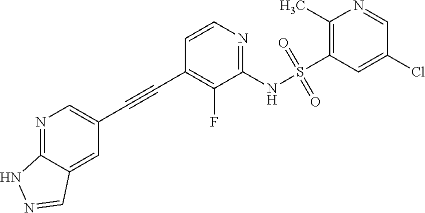 [SMILES: Cc1ncc(Cl)cc1S(=O)(=O)Nc1nccc(C#Cc2cnc3[nH]ncc3c2)c1F] | 14.03 (s, 1H), 8.76-8.66 (m, 2H), 8.59 (d, J = 1.9 Hz, 1H), 8.33 (d, J = 2.5 Hz, 1H), 8.25 (d, J = 1.3 Hz, 1H), 7.86 (s, 1H), 7.09 (s, 1H), 2.78 (s, 3H). | Method C RT: 3.247 min m/z [M − H]⁺ 443.09 |
| 5 | 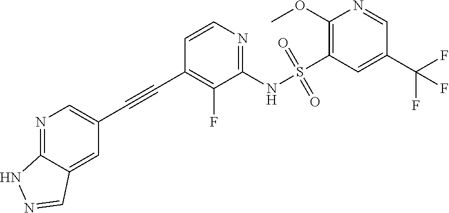 [SMILES: COc1ncc(C(F)(F)F)cc1S(=O)(=O)Nc1nccc(C#Cc2cnc3[nH]ncc3c2)c1F] | 14.03 (s, 1H), 8.81 (s, 1H), 8.73 (d, J = 2.0 Hz, 1H), 8.58 (s, 1H), 8.43 (s, 1H), 8.25 (d, J = 1.3 Hz, 1H), 7.83 (s, 1H), 7.09 (s, 1H), 3.97 (s, 3H). | Method C RT: 3.720 min m/z [M − H]⁺ 493.10 |
| 6 | 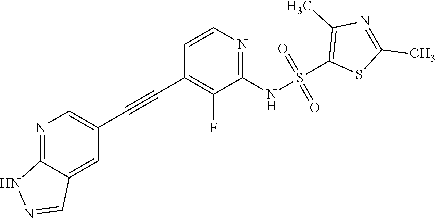 [SMILES: Cc1nc(C)c(S(=O)(=O)Nc2nccc(C#Cc3cnc4[nH]ncc4c3)c2F)s1] | 13.98 (s, 1H), 8.71 (d, J = 2.0 Hz, 1H), 8.56 (d, J = 2.0 Hz, 1H), 8.23 (d, J = 1.3 Hz, 1H), 7.82 (s, 1H), 6.85 (s, 2H), 2.56 (s, 3H), 2.47 (s, 3H). | Method D RT: 2.280 min m/z [M − H]⁺ 429.10 |
| 7 | 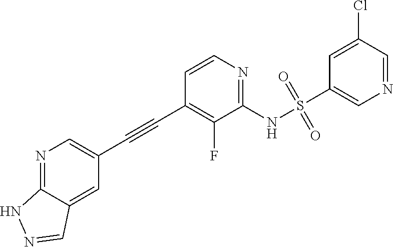 [SMILES: O=S(=O)(Nc1nccc(C#Cc2cnc3[nH]ncc3c2)c1F)c1cncc(Cl)c1] | 14.03 (s, 1H), 9.05 (d, J = 2.0 Hz, 1H), 8.91 (d, J = 2.4 Hz, 1H), 8.74 (d, J = 2.0 Hz, 1H), 8.60 (d, J = 2.0 Hz, 1H), 8.42 (t, J = 2.1 Hz, 1H), 8.25 (s, 1H), 8.01 (s, 1H), 7.26 (s, 1H). | Method D RT: 2.490 min m/z [M − H]⁺ 429.11 |
| 8 | 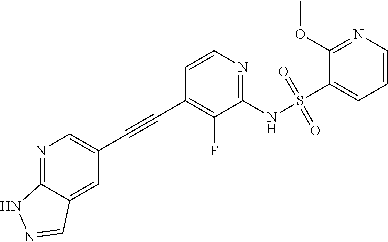 [SMILES: COc1ncccc1S(=O)(=O)Nc1nccc(C#Cc2cnc3[nH]ncc3c2)c1F] | 14.04 (s, 1H), 11.47 (s, 1H), 8.74 (d, J = 2.0 Hz, 1H), 8.60 (d, J = 2.0 Hz, 1H), 8.40 (s, 1H), 8.25 (d, J = 1.4 Hz, 2H), 7.95 (s, 1H), 7.38-7.15 (m, 2H), 3.91 (s, 3H). | Method B RT: 1.750 min m/z [M − H]⁺ 425.13 |
| 9 | 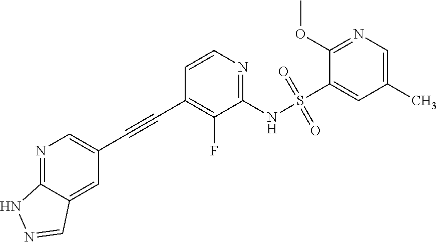 [SMILES: COc1ncc(C)cc1S(=O)(=O)Nc1nccc(C#Cc2cnc3[nH]ncc3c2)c1F] | 14.03 (s, 1H), 11.43 (s, 1H), 8.74 (d, J = 2.0 Hz, 1H), 8.60 (d, J = 2.0 Hz, 1H), 8.25 (d, J = 1.3 Hz, 1H), 8.21 (s, 1H), 8.08 (s, 1H), 7.94 (s, 1H), 7.25 (s, 1H), 3.85 (s, 3H), 2.30 (s, 3H). | Method B RT: 2.030 min m/z [M − H]⁺ 439.13 |
| 10 | 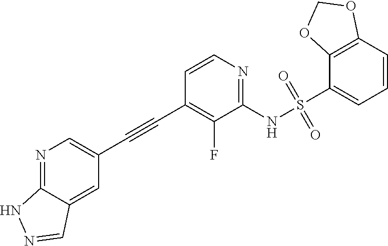 [SMILES: O=S(=O)(Nc1nccc(C#Cc2cnc3[nH]ncc3c2)c1F)c1cccc2c1OCO2] | 14.02 (s, 1H), 11.55 (s, 1H), 8.74 (d, J = 2.0 Hz, 1H), 8.60 (d, J = 2.0 Hz, 1H), 8.25 (d, J = 1.4 Hz, 1H), 8.01 (s, 1H), 7.28 (dd, J = 8.3, 1.1 Hz, 2H), 7.16 (d, J = 7.7 Hz, 1H), 6.98 (t, J = 8.0 Hz, 1H), 6.15 (s, 2H). | Method B RT: 1.997 min m/z [M − H]⁺ 438.14 |
| 11 | 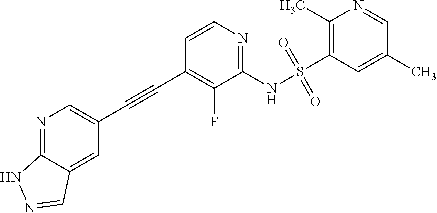 [SMILES: Cc1cnc(C)c(S(=O)(=O)Nc2nccc(C#Cc3cnc4[nH]ncc4c3)c2F)c1] | 13.95 (s, 1H), 12.64 (s, 1H), 8.69 (s, 1H), 8.52 (s, 1H), 8.21 (s, 2H), 8.14 (s, 1H), 8.02 (s, 1H), 7.56 (s, 1H), 6.57 (s, 1H), 2.73-2.64 (m, 3H), 2.30 (s, 3H). | Method E RT: 2.053 min m/z [M − H]⁺ 423.04 |
| 12 | 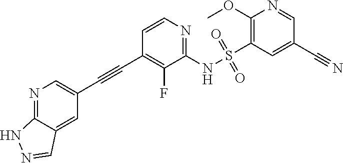 [SMILES: COc1ncc(C#N)cc1S(=O)(=O)Nc1nccc(C#Cc2cnc3[nH]ncc3c2)c1F] | 14.03 (s, 1H), 11.84 (s, 1H), 8.92 (s, 1H), 8.74 (d, J = 2.0 Hz, 1H), 8.65 (s, 1H), 8.60 (d, J = 2.0 Hz, 1H), 8.25 (d, J = 1.3 Hz, 1H), 7.95 (s, 1H), 7.23 (d, J = 16.4 Hz, 1H), 3.99 (s, 3H). | Method B RT: 1.960 min m/z [M − H]⁺ 450.20 |
| 13 | 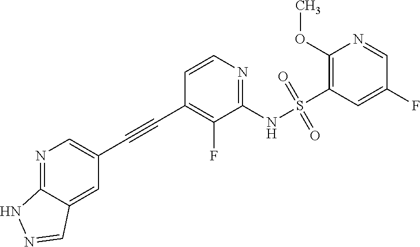 [SMILES: COc1ncc(F)cc1S(=O)(=O)Nc1nccc(C#Cc2cnc3[nH]ncc3c2)c1F] | 14.03 (s, 1H), 11.67 (s, 1H), 8.74 (d, J = 2.0 Hz, 1H), 8.60 (d, J = 2.0 Hz, 1H), 8.46 (s, 1H), 8.26 (d, J = 1.4 Hz, 1H), 8.18 (s, 1H), 7.98 (s, 1H), 7.31 (s, 1H), 3.90 (s, 3H). | Method B RT: 2.090 min m/z [M − H]⁺ 443.13 |
| 14 | 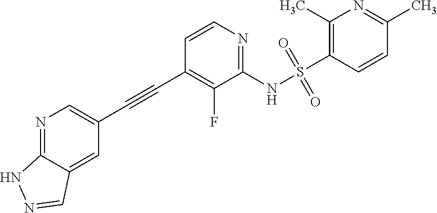 [SMILES: Cc1ccc(S(=O)(=O)Nc2nccc(C#Cc3cnc4[nH]ncc4c3)c2F)c(C)n1] | 13.94 (s, 1H), 8.68 (d, J = 2.0 Hz, 1H), 8.51 (d, J = 2.0 Hz, 1H), 8.30 (s, 2H), 8.21 (s, 1H), 8.04 (d, J = 8.0 Hz, 1H), 7.49 (d, J = 5.1 Hz, 1H), 7.06 (d, J = 7.9 Hz, 1H), 6.47 (t, J = 4.6 Hz, 1H), 2.67 (s, 3H), 2.40 (s, 3H). | Method E RT: 1.880 min m/z [M − H]⁺ 423.02 |
| 15 | 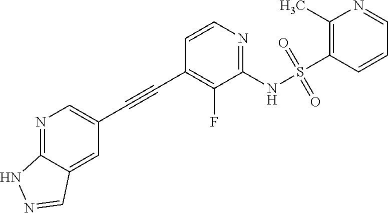 [SMILES: Cc1ncccc1S(=O)(=O)Nc1nccc(C#Cc2cnc3[nH]ncc3c2)c1F] | 8.67 (d, J = 2.0 Hz, 1H), 8.51 (d, J = 2.0 Hz, 1H), 8.37 (dd, J = 4.8, 1.8 Hz, 1H), 8.33 (s, 2H), 8.21 (s, 1H), 8.15 (dd, J = 7.8, 1.8 Hz, 1H), 7.49 (d, J = 5.1 Hz, 1H), 7.23 (dd, J = 7.8, 4.8 Hz, 1H), 6.51-6.45 (m, 1H), 2.72 (s, 3H). | Method D RT: 1.977 min m/z [M − H]⁺ 409.14 |
| 16 | 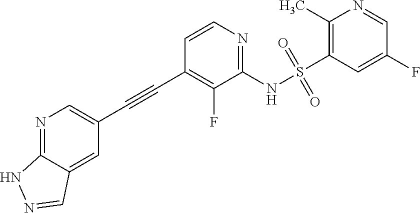 [SMILES: Cc1ncc(F)cc1S(=O)(=O)Nc1nccc(C#Cc2cnc3[nH]ncc3c2)c1F] | 14.04 (s, 1H), 12.14 (s, 1H), 8.73 (dd, J = 5.4, 2.4 Hz, 2H), 8.60 (d, J = 2.0 Hz, 1H), 8.26 (s, 1H), 8.21 (dd, J = 8.4, 2.9 Hz, 1H), 7.94 (s, 1H), 7.24 (s, 1H), 2.80 (d, J = 1.2 Hz, 3H). | Method F RT: 4.063 min m/z [M − H]⁺ 426.98 |
| 17 | 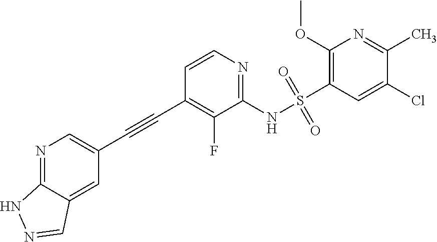 [SMILES: COc1nc(C)c(Cl)cc1S(=O)(=O)Nc1nccc(C#Cc2cnc3[nH]ncc3c2)c1F] | 14.03 (s, 1H), 11.59 (s, 1H), 8.74 (d, J = 2.0 Hz, 1H), 8.60 (d, J = 2.0 Hz, 1H), 8.25 (d, J = 1.3 Hz, 1H), 8.16 (s, 1H), 8.00 (s, 1H), 7.32 (s, 1H), 3.91 (s, 3H), 2.54 (s, 3H). | Method D RT: 3.150 min m/z [M − H]⁺ 473.08 |
| 18 | 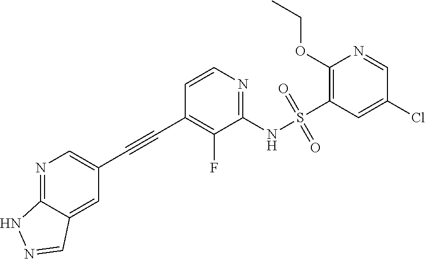 [SMILES: CCOc1ncc(Cl)cc1S(=O)(=O)Nc1nccc(C#Cc2cnc3[nH]ncc3c2)c1F] | 14.04 (s, 1H), 11.64 (s, 1H), 8.75 (d, J = 2.0 Hz, 1H), 8.61 (d, J = 2.0 Hz, 1H), 8.47 (s, 1H), 8.26 (d, J = 1.3 Hz, 2H), 7.97 (s, 1H), 7.31 (s, 1H), 4.37 (s, 2H), 1.26-0.97 (m, 3H). | Method B RT: 2.703 min m/z [M − H]⁺ 473.06 |
| 19 | 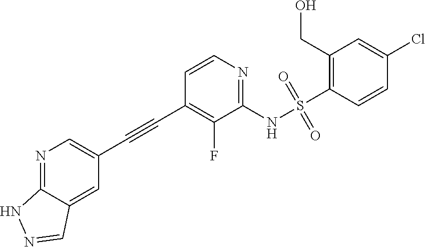 [SMILES: O=S(=O)(Nc1nccc(C#Cc2cnc3[nH]ncc3c2)c1F)c1ccc(Cl)cc1CO] | 14.02 (s, 1H), 11.66 (s, 1H), 8.73 (d, J = 2.0 Hz, 1H), 8.59 (s, 1H), 8.25 (s, 1H), 8.00 (d, J = 8.4 Hz, 2H), 7.76 (s, 1H), 7.54 (d, J = 8.2 Hz, 1H), 7.25 (s, 1H), 5.60 (s, 1H), 4.96 (s, 2H). | Method G RT: 3.007 min m/z [M − H]⁺ 458.03 |
| 20 | 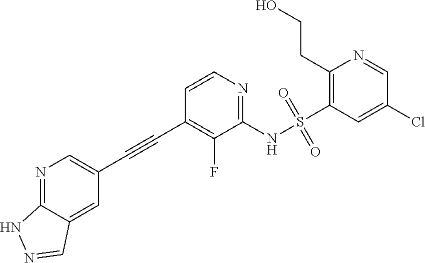 [SMILES: O=S(=O)(Nc1nccc(C#Cc2cnc3[nH]ncc3c2)c1F)c1cc(Cl)cnc1CCO] | 13.95 (s, 1H), 8.68 (d, J = 1.9 Hz, 1H), 8.51 (dd, J = 8.4, 2.3 Hz, 2H), 8.27 (s, 1H), 8.24-8.18 (m, 2H), 7.54 (d, J = 5.1 Hz, 1H), 6.55 (t, J = 4.6 Hz, 1H), 4.52 (s, 1H), 3.70 (t, J = 7.4 Hz, 2H), 3.37 (t, J = 7.4 Hz, 2H). | Method D RT: 2.277 min m/z [M − H]⁺ 473.06 |

### Intermediate 1

### Step 1:3-(benzylsulfanyl)-2,5-dimethylpyridine

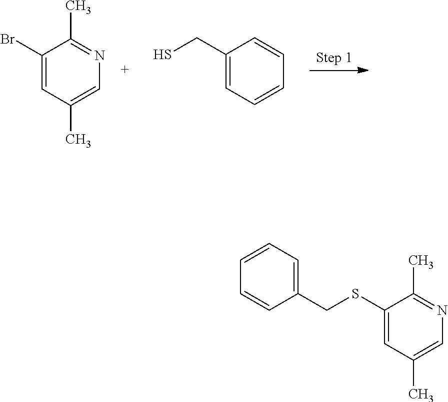

3-Bromo-2,5-dimethylpyridine (300 mg, 1.612 mmol, 1.0 eq.), benzyl mercaptan (240 mg, 1.935 mmol, 1.2 eq.) and DIPEA (0.702 ml, 4.031 mmol, 2.5 eq.) were dissolved in anh. toluene (15 ml). After degassing with argon for 15 min, xantphos (187 mg, 0.322 mmol, 0.2 eq.) and Pd₂(dba)₃ (148 mg, 0.161 mmol, 0.1 eq.) were added and the resulting mixture was stirred in a microwave for 1 h at 150° C. Afterward, insolubilities were filtered off, the solvent was evaporated in vacuo and the residue was purified by FC (SiO₂, 0-20% EA in hexane) affording 3-(benzylsulfanyl)-2,5-dimethylpyridine (380 mg, Y: 98%) as an oily product.

¹H NMR (300 MHZ, CDCl₃) δ 8.17-8.13 (m, 1H), 7.34-7.27 (m, 6H), 4.08 (s, 2H), 2.53 (s, 3H), 2.26 (s, 3H).

MS m/z: [M+H]⁺ 230.50

### Step 2:2,5-Dimethylpyridine-3-sulfonyl chloride

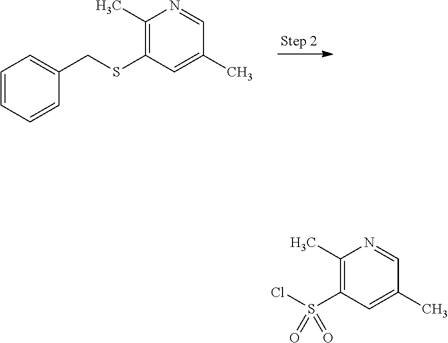 [SMILES: Cc1cnc(C)c(SCc2ccccc2)c1 ; Cc1cnc(C)c(S(=O)(=O)Cl)c1]

3-(Benzylsulfanyl)-2,5-dimethylpyridine (380 mg, 1.574 mmol, 1.0 eq.) was dissolved in a mixture of ACN (10 ml), acetic acid (0.6 ml) and water (0.4 ml). The mixture was cooled to 0-5° C. in an ice bath. Next, 1,3-dichloro-5,5-dimethylhydantoin (620 mg, 3.148 mmol, 2.0 eq.) was added portionwise over 10 minutes and the reaction was stirred at the same temperature for 2 h. Afterwards, the mixture was evaporated and the slurry residue was divided between water and EA. The aqueous layer was additionally extracted with EA. Organic layers were combined, dried over sodium sulphate, filtered and evaporated. The crude product was purified by FC (SiO₂, 0-10% EA in hexane), affording 2,5-dimethylpyridine-3-sulfonyl chloride (255 mg, Y: 75%) as an oily product which was used in the synthesis of Example 11.

¹H NMR (300 MHZ, CDCl₃) δ 8.64 (d, J=2.1 Hz, 1H), 8.15 (d, J=2.1 Hz, 1H), 2.98 (s, 3H), 2.47 (m, 3H).

Intermediates 2-10 were synthesized using methods analogous to those described above:

| | | | Used in the |
|---|---|---|---|
| | | | synthesis of |
| Intermediate | Structure | Analytical Data | Example |
| 2 | 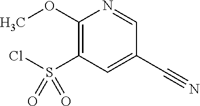 | ¹H NMR (400 MHz, DMSO- d₆) δ 8.78 (d, J = 2.2 Hz, 1H), 8.51 (d, J = 2.2 Hz, 1H), 4.31 (s, 3H) | 12 |
| 3 | 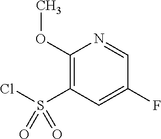 | ¹H NMR (400 MHz, DMSO- d₆) δ 8.40 (d, J = 3.0 Hz, 1H), 8.07 (dd, J = 6.6, 3.0 Hz, 1H), 4.22 (s, 3H) | 13 |
| 4 | 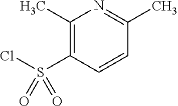 | MS m/z: [M + H]⁺ 206.00 | 14 |
| 5 | 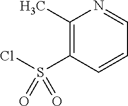 | MS m/z: [M + H]⁺ 191.90 | 15 |
| 6 | 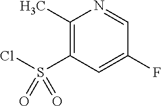 | ¹H NMR (300 MHz, DMSO- d₆) δ 8.74 (dd, J = 2.9, 1.5 Hz, 1H), 8.21 (d, J = 2.9 Hz, 1H), 2.80 (d, J = 1.1 Hz, 3H). MS m/z: [M + H]⁺ 210.00 | 16 |
| 7 | 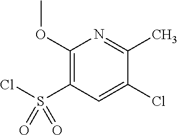 | ¹H NMR (300 MHz, DMSO- d₆) δ 7.85 (s, 1H), 3.84 (s, 3H), 2.44 (s, 3H). | 17 |
| 8 | 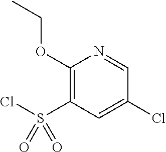 | ¹H NMR (300 MHz, CDCl₃) δ 8.42 (d, J = 2.5 Hz, 1H), 8.22 (d, J = 2.6 Hz, 1H), 4.65 (q, J = 7.1 Hz, 2H), 1.52 (t, J = 7.1 Hz, 3H). | 18 |
| 9 | 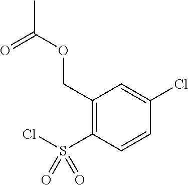 [SMILES: CC(=O)OCc1cc(Cl)ccc1S(=O)(=O)Cl] | ¹H NMR (300 MHz, CDCl₃) δ 8.08 (d, J = 8.6 Hz, 1H), 7.68 (dt, J = 1.9, 0.9 Hz, 1H), 7.55 (dd, J = 8.6, 2.2 Hz, 1H), 5.62 (s, 2H), 2.25 (s, 3H). | 19 |
| 10 | 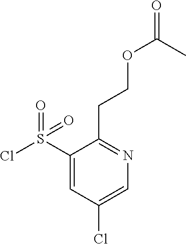 [SMILES: CC(=O)OCCc1ncc(Cl)cc1S(=O)(=O)Cl] | ¹H NMR (300 MHz, DMSO- d₆) δ 8.54 (d, J = 2.5 Hz, 1H), 8.03 (d, J = 2.5 Hz, 1H), 4.41 (t, J = 7.2 Hz, 2H), 3.47 (t, J = 7.2 Hz, 2H), 1.98 (s, 3H). | 20 |

### Biological and Pharmacokinetic Testing

### (a) GCN2 Enzyme Inhibition

### Assay Protocol Overview:

The inhibitory activity of the Example compounds towards GCN2 enzyme was measured according to the description below, using a LanthaScreen TR-FRET (Time Resolved Fluorescence Resonance Energy Transfer) Kinase Activity assay distributed by ThermoFisher Scientific.

The full-length human GCN2 enzyme (UniProt accession number Q9P2K8) was used for all experiments (Carna Bioscience). The TR-FRET pair was composed of GFP-elF2a and LanthaScreen Terbium-labeled anti-pelF2a (pSer52) Antibody.

Each example compound was dissolved in DMSO (0.15 mM) and dispensed in a 384-well plate by a D300 dispenser (Tecan) to a final concentration range 3000-0.13 nM using the logarithmic dilution mode in 2 replicates. Full inhibition (3000 nM commercial reference inhibitor) and DMSO vehicle control wells were also included on the same plate. All volumes were normalized to the final DMSO concentration of 2% of the reaction volume. Next, 5 μL of H₂O was added to each well of the plate.

The enzyme mixture was prepared to obtain the following concentrations:
GCN2—30 nM
unloaded tRNA—0.3 nM
HEPES (pH=7.0)—100 mM
MgCl₂—20 mM; MnCl₂—10 mM

The mixture was applied by adding 5 μL to each well of the plate. The enzyme and the tested compound were then incubated at room temperature for 20 min while shaking at 450 rpm.

The substrate mixture was prepared to obtain the following concentrations:
GFP-elF2α—240 nM
ATP-30 PM
HEPES (pH=7.0)—50 mM
MgCl₂—10 mM
MnCl₂—5 mM.

The mixture was applied by adding 5 μL to each well of the plate. Thus, the final concentrations of the 15 μL reaction mixture were as follows:
GCN2—10 nM
unloaded tRNA—0.1 nM
GFP-elF2α—80 nM
ATP-10 μM
HEPES (1 M, pH=7.0)—50 mM
MgCl₂—10 mM
MnCl₂—5 mM.

The reaction was allowed to proceed at room temperature for 30 min while shaking at 450 rpm.

The antibody mixture was prepared to obtain the following concentrations:
Na₂EDTA·2H₂O—40 mM, in TR-FRET Dilution Buffer (Life technologies)
Tb-anti-pelF2a antibodies—4 nM.

The mixture was applied by adding 15 μL to each well. The plate was then incubated at room temperature for 60 min while shaking at 450 rpm and then read using Tecan Spark reader using specific TR-FRET filters.

The analysis of the GFP/Tb fluorescence results was conducted with GraphPad Prism to determine IC₅₀ for each of the example compounds using 4-parameter model: log (inhibitor) vs. response-variable slope. IC₅₀ and Kᵢ values were calculated in the usual way. The assay was carried out between 1 and 3 times. The results in the table below are the mean results from replicate assays for the compound in question, where applicable.

### Results

TABLE 1

| Example # | GCN2 Ki (nM) 10 μM ATP |
|---|---|
| 1 | 172.50 |
| 2 | 107.10 |
| 3 | 127.90 |
| 4 | 2.49 |
| 5 | 5.78 |
| 6 | 29.31 |
| 7 | 127.20 |
| 8 | 59.87 |
| 9 | 6.60 |
| 10 | 209.00 |
| 11 | 8.17 |
| 12 | 7.76 |
| 13 | 4.42 |
| 14 | 17.76 |
| 15 | 123.80 |
| 16 | 4.19 |
| 17 | 2.28 |
| 18 | 8.83 |
| 19 | 64.95 |
| 20 | 66.98 |

The results in Table 1 show that the compounds of the invention are potent inhibitors of GCN2.

### (b) Kinetic Solubility

### Assay Protocol Overview:

The kinetic solubility assay investigates a solubility based on the amount of material which remains in solution after a precipitation process. Compounds for kinetic solubility test are prepared as 10 mM stock solutions in DMSO. Assay is performed using Multiscreen Vacuum Manifold. A buffer of interest (in standard protocol PBS buffer at pH=7.4 is used) is spiked with stock solution and incubated for 90 minutes at room temperature. After that time solution/suspension is filtrated. The concentration of each compound is determined on the base of prepared calibration curve using UV-VIS spectrophotometry method. The assay is made in triplicate.

Buffer of interest: 0.24 g of KH₂PO₄, 1.44 g of Na₂HPO₄, 0.2 g of KCl and 8 g NaCl and dissolve in 1 L distilled H₂O; adjust pH to appropriate value (pH 7.4).

190 μl of buffer of interest is dispensed into the wells of a 96 well filter plate, followed by 10 μl of compound (10 mM stock solution in DMSO). The plate is shaken gently at room temperature for 90 minutes at 500 rpm using a BioSan, Plate Shaker-Thermostat, PST-60HL-4. After 90 minutes, the plate is filtered using a vacuum manifold and vacuum pump. 100 μl of each filtrate and 100 μl acetonitrile is transferred to a 96 well UV-visible light transparent plate and the UV-visible absorption spectrum is measured using a Biotek Synergy 2 multiplate reader from 250-500 nm, interval range 10 nm. The amount of test compound is calculated using a calibration curve prepared by serial dilution of compounds in equivalent amounts of DMSO and acetonitrile.

The compounds of the invention have both strong GCN2 inhibitory activity and good kinetic solubility as measured in the assays described above.

1. A compound of formula (I), or a pharmaceutically acceptable ester, amide, carbamate or salt thereof, including a pharmaceutically acceptable salt of such an ester, amide or carbamate: 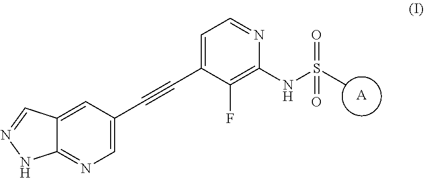
wherein A is selected from the group consisting of: 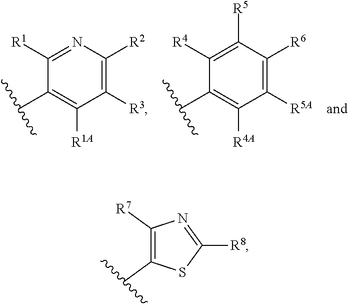
wherein R¹, R^{1A}, R² and R³ are each independently selected from the group consisting of hydrogen; halogen, cyano; C₁₋₂alkyl, optionally substituted by 1, 2 or 3 groups independently selected from the group consisting of halogen and OH; and O—C₁₋₂alkyl; and
wherein R⁴, R^{4A}, R⁵, R^{5A} and R⁶ are each independently selected from the group consisting of hydrogen; halogen, cyano; C₁₋₂alkyl, optionally substituted by 1, 2 or 3 groups independently selected from the group consisting of halogen and OH; and O—C₁₋₂alkyl; or
R⁴ and R⁵ together with the 2 atoms in the A ring to which they are attached form a 5- or 6-membered ring containing 2 oxygen atoms, in which the 2 oxygen atoms are directly attached to the 2 atoms in the A ring; and R_{1A}, R_{2A} and R₃ are each independently selected from the group consisting of hydrogen; halogen, cyano; C₁₋₂alkyl, optionally substituted by 1, 2 or 3 groups independently selected from the group consisting of halogen and OH; and O—C₁₋₂alkyl; and
wherein R⁷ and R⁸ are each independently selected from the group consisting of hydrogen; halogen, cyano; C₁₋₂alkyl, optionally substituted by 1, 2 or 3 groups independently selected from the group consisting of halogen and OH; and O—C₁₋₂alkyl;
and wherein A is not:

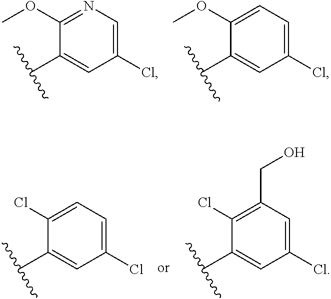

2. The compound as claimed in claim 1, wherein A is 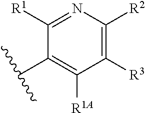
wherein R^{1A}, R¹, R² and R³ are each independently selected from the group consisting of hydrogen; halogen, cyano; C₁₋₂alkyl, optionally substituted by 1, 2 or 3 groups independently selected from the group consisting of halogen and OH; and O—C₁₋₂alkyl;
and wherein A is not

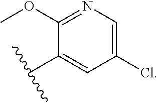 [SMILES: *c1cc(Cl)cnc1OC]

3. The compound as claimed in claim 2, wherein:
R^{1A} is hydrogen
R¹ is selected from the group consisting of hydrogen, methyl, —CH₂CH₂OH and O—C₁₋₂alkyl;
R² is selected from the group consisting of hydrogen and methyl; and
R³ is selected from the group consisting of hydrogen, fluorine, chlorine, cyano, methyl and trifluoromethyl.

4. The compound as claimed in claim 3, wherein:
R¹ is selected from the group consisting of methyl, —CH₂CH₂OH and O—C₁₋₂alkyl;
R² is selected from the group consisting of hydrogen and methyl; and
R³ is selected from the group consisting of hydrogen, fluorine, chlorine, cyano, methyl and trifluoromethyl.

5. The compound as claimed in claim 4, wherein:
R¹ is selected from the group consisting of methyl and O—C₁₋₂alkyl;
R² is selected from the group consisting of hydrogen and methyl; and
R³ is selected from the group consisting of fluorine, chlorine, cyano, methyl and trifluoromethyl.

6. The compound as claimed in claim 1, wherein A is 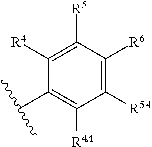
wherein R⁴, R^{4A}, R⁵, R^{5A} and R⁶ are each independently selected from the group consisting of hydrogen; halogen, cyano; C₁₋₂alkyl, optionally substituted by 1, 2 or 3 groups independently selected from the group consisting of halogen and OH; and O—C₁₋₂alkyl; or
wherein R⁴ and R⁵ together with the 2 atoms in the A ring to which they are attached form a 5- or 6-membered ring containing 2 oxygen atoms, in which the 2 oxygen atoms are directly attached to the 2 atoms in the A ring; and R^{4A}, R^{5A} and R⁶ are each independently selected from the group consisting of hydrogen; halogen, cyano; C₁₋₂alkyl, optionally substituted by 1, 2 or 3 groups independently selected from the group consisting of halogen and OH; and O—C₁₋₂alkyl;
and wherein A is not

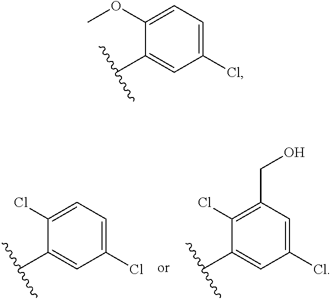

7. The compound as claimed in claim 6, wherein:
R^{4A} and R^{5A} are hydrogen;
R⁴ is selected from the group consisting of hydrogen, fluorine and —CH₂OH;
R⁵ is selected from the group consisting of hydrogen, cyano and —OCH₃; and
R⁶ is selected from the group consisting of hydrogen and chlorine;
or wherein:
R^{4A} and R^{5A} are hydrogen;
R⁴ and R⁵ together with the 2 carbon atoms in the A ring to which they are attached form a 1,3-dioxalane ring; and
R⁶ is selected from the group consisting of hydrogen and chlorine.

8. The compound as claimed in claim 7, wherein:
R⁴ is-CH₂OH; and
R⁵ is hydrogen; and
R⁶ is chlorine.

9. The compound as claimed in claim 1, wherein A is 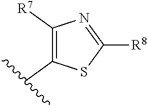
wherein R⁷ and R⁸ are independently selected from the group consisting of hydrogen; halogen, cyano; C₁₋₂alkyl, optionally substituted by 1, 2 or 3 groups independently selected from the group consisting of halogen and OH; and O—C₁₋₂alkyl.

10. The compound as claimed in claim 9, wherein R₇ and R₈ are methyl.

11. The compound as claimed in claim 1, wherein the compound of formula (I) is selected from the group consisting of:
2-Fluoro-N-[3-fluoro-4-(2-{1H-pyrazolo[3,4-b]pyridin-5-yl}ethynyl)pyridin-2-yl]-3-methoxybenzene-1-sulfonamide (example 1);
3-Cyano-N-[3-fluoro-4-(2-{1H-pyrazolo[3,4-b]pyridin-5-yl}ethynyl)pyridin-2-yl]benzene-1-sulfonamide (example 2);
N-{3-Fluoro-4-[2-(1H-indazol-5-yl)ethynyl]pyridin-2-yl}-3-methoxybenzene-1-sulfonamide (example 3);
5-Chloro-N-[3-fluoro-4-(2-{1H-pyrazolo[3,4-b]pyridin-5-yl}ethynyl)pyridin-2-yl]-2-methylpyridine-3-sulfonamide (example 4);
N-[3-Fluoro-4-(2-{1H-pyrazolo[3,4-b]pyridin-5-yl}ethynyl)pyridin-2-yl]-2-methoxy-5-(trifluoromethyl)pyridine-3-sulfonamide (example 5);
N-[3-Fluoro-4-(2-{1H-pyrazolo[3,4-b]pyridin-5-yl}ethynyl)pyridin-2-yl]-2,4-dimethyl-1,3-thiazole-5-sulfonamide (example 6);
5-Chloro-N-[3-fluoro-4-(2-{1H-pyrazolo[3,4-b]pyridin-5-yl}ethynyl)pyridin-2-yl]pyridine-3-sulfonamide (example 7);
N-[3-Fluoro-4-(2-{1H-pyrazolo[3,4-b]pyridin-5-yl}ethynyl)pyridin-2-yl]-2-methoxypyridine-3-sulfonamide (example 8);
N-[3-Fluoro-4-(2-{1H-pyrazolo[3,4-b]pyridin-5-yl}ethynyl)pyridin-2-yl]-2-methoxy-5-methyl-pyridine-3-sulfonamide (example 9);
N-[3-Fluoro-4-(2-{1H-pyrazolo[3,4-b]pyridin-5-yl}ethynyl)pyridin-2-yl]-2H-1,3-benzodioxole-4-sulfonamide (example 10);
N-[3-Fluoro-4-(2-{1H-pyrazolo[3,4-b]pyridin-5-yl}ethynyl)pyridin-2-yl]-2,5-dimethylpyridine-3-sulfonamide (example 11);
5-Cyano-N-[3-fluoro-4-(2-{1H-pyrazolo[3,4-b]pyridin-5-yl}ethynyl)pyridin-2-yl]-2-methoxypyridine-3-sulfonamide (example 12);
5-Fluoro-N-[3-fluoro-4-(2-{2H-pyrazolo[4,3-b]pyridin-5-yl}ehynyl)pyridin-2-yl]-2-methoxypyridine-3-sulfonamide (example 13);
N-[3-Fluoro-4-(2-{1H-pyrazolo[3,4-b]pyridin-5-yl}ethynyl)pyridin-2-yl]-2,6-dimethylpyridine-3-sulfonamide (example 14);
5-Fluoro-N-[3-fluoro-4-(2-{2H-pyrazolo[4,3-b]pyridin-5-yl}ethynyl)pyridin-2-yl]-2-methoxypyridine-3-sulfonamide (example 15);
5-Fluoro-N-[3-fluoro-4-(2-{1H-pyrazolo[3,4-b]pyridin-5-yl}ethynyl)pyridin-2-yl]-2-methylpyridine-3-sulfonamide (example 16);
5-Chloro-2-ethoxy-N-[3-fluoro-4-(2-{1H-pyrazolo[3,4-b]pyridin-5-yl}ethynyl)-pyridin-2-yl]pyridine-3-sulfonamide (example 17);
5-Chloro-2-ethoxy-N-[3-fluoro-4-(2-{1H-pyrazolo[3,4-b]pyridin-5-yl}ethynyl)-pyridin-2-yl]pyridine-3-sulfonamide (example 18);
N-[3-Fluoro-4-(2-{1H-pyrazolo[3,4-b]pyridin-5-yl}ethynyl)pyridin-2-yl]-2,5-dimethylpyridine-3-sulfonamide (example 19); and
5-chloro-N-[3-fluoro-4-(2-{1H-pyrazolo[3,4-b]pyridin-5-yl}ethynyl)pyridin-2-yl]-2-(2-hydroxyethyl)pyridine-3-sulfonamide (example 20)

12. A pharmaceutical composition comprising the compound according to claim 1 and at least one pharmaceutically acceptable carrier or excipient.

13. The pharmaceutical composition according to claim 12, wherein said composition further comprises at least one further therapeutic agent.

14. The pharmaceutical composition according to claim 13, wherein the further therapeutic agent is 1-asparaginase or a proteasome inhibitor.

15. The compound according to, claim 1, for use as a medicament.

16. The compound according to, claim 1, for use in the treatment or prophylaxis of a disease or disorder in which the inhibition of GCN2 provides a therapeutic effect.

17. The compound according to, claim 1, for use in the treatment or prophylaxis of a disease or disorder selected from the group consisting of: cancer (for example solid cancers and haematological cancers), diabetic retinopathy, myocardial ischemia, diabetic cardiomyopathy, allergic airway inflammation, doxorubicin-induced cardiotoxicity and nonalcoholic fatty liver disease (NAFLD).

18. The compound for use according to claim 16, wherein the disease or disorder is a cancer, and the cancer is selected from the group consisting of colorectal cancer (e.g., colorectal cancer, rectal cancer, anal cancer, familial colorectal cancer, hereditary nonpolyposis colorectal cancer, gastrointestinal stromal tumor), lung cancer (e.g., non-small cell lung cancer, small cell lung cancer, malignant mesothelioma), mesothelioma, pancreatic cancer (e.g., pancreatic duct cancer, pancreatic endocrine tumor), pharyngeal cancer, laryngeal cancer, esophagus cancer, gastric cancer (e.g., papillary adenocarcinoma, mucinous adenocarcinoma, adenosquamous carcinoma), duodenal cancer, small intestinal cancer, breast cancer (e.g., invasive ductal carcinoma, ductal carcinoma in situ, inflammatory breast cancer), ovarian cancer (e.g., ovarian epithelial carcinoma, extragonadal germ cell tumor, ovarian germ cell tumor, ovarian low malignant potential tumor), testis tumor, prostate cancer (e.g., hormone-dependent prostate cancer, non-hormone dependent prostate cancer, castration-resistant prostate cancer), liver cancer (e.g., hepatoma, primary liver cancer, extrahepatic bile duct cancer), thyroid cancer (e.g., medullary thyroid carcinoma), renal cancer (e.g., renal cell carcinoma (e.g., clear cell renal cell carcinoma), transitional cell carcinoma of renal pelvis and ureter), uterine cancer (e.g., cervixcancer, uterine body cancer, uterus sarcoma), gestational choriocarcinoma, brain tumor (e.g., medulloblastoma, glioma, glioblastoma, pineal astrocytoma, pilocytic astrocytoma, diffuse astrocytoma, anaplastic astrocytoma, hypophyseal adenoma), retina blastoma, skin cancer (e.g., basal cell carcinoma, malignant melanoma (melanoma)), sarcoma (e.g., rhabdomyosarcoma, leiomyosarcoma, soft tissue sarcoma, spindle cell sarcoma, osteosarcoma), malignant bone tumor, urinary bladder cancer, and hematologic cancer (e.g., multiple myeloma, smouldering myeloma, plasmacytoma, leukemia (e.g., acute myeloid leukemia, acute lymphocytic leukemia (including blast crisis of chronic leukemia)), non-Hodgkin's lymphoma, malignant lymphoma, Hodgkin's disease, chronic myeloproliferative disease), and cancer of unknown primary nucleus); and/or
wherein the disease or disorder is a cancer having a MYC mutation (i.e. a cancer in which there is a mutation in the MYC gene).

19. A method for the treatment or prophylaxis of a disease or disorder in which the inhibition of GCN2 provides a therapeutic effect in a mammal, which comprises administering to the mammal a therapeutically effective amount the composition according to claim 12, for example a disease or disorder selected from the group consisting of: cancer (for example solid cancers and haematological cancers), diabetic retinopathy, myocardial ischemia, diabetic cardiomyopathy, allergic airway inflammation, doxorubicin-induced cardiotoxicity and nonalcoholic fatty liver disease (NAFLD).

20. A use of the compound according to claim 1 for the manufacture of a medicament for the treatment or prophylaxis of a disease or disorder in which the inhibition of GCN2 provides a therapeutic effect, for example a disease or disorder selected from the group consisting of: cancer (for example solid cancers and haematological cancers), diabetic retinopathy, myocardial ischemia, diabetic cardiomyopathy, allergic airway inflammation, doxorubicin-induced cardiotoxicity and nonalcoholic fatty liver disease (NAFLD).
